(19) United States
(12) Patent Application Publication
(10) Pub. No.: US 2026/0054025 A1
(43) Pub. Date: Feb. 26, 2026

(54) MEDICAL TUBES AND CONNECTORS FOR BREATHING CIRCUITS

(71) Applicant: Fisher & Paykel Healthcare Limited, Auckland (NZ)

(72) Inventors: Matthew Liam BUSWELL, Auckland (NZ); Alexander Jordan PEPPERELL, Auckland (NZ); David VAN BOKHOVEN, Auckland (NZ)

(21) Appl. No.: 18/871,654

(22) PCT Filed: Jun. 16, 2023
(86) PCT No.: PCT/IB2023/056241

§ 371 Date: Dec. 4, 2024

Related U.S. Application Data
(60) Provisional application No. 63/366,611, filed on Jun. 17, 2022.

(51) Int. Cl. A61M 16/08; A61M 16/10

(52) U.S. Cl. CPC A61M 16/0875; A61M 16/0808; A61M 16/0816; A61M 16/1095

(57) ABSTRACT

Disclosed is medical tube assembly, the medical tube assembly comprising: a connector, a collar, and a tube, the tube may be made of a water absorbing material. The tube comprising a main body portion and an increasing gases passage cross-sectional area portion. The collar is configured to engage an external surface of the tube when the connector is connected to the tube, and the tube is retained between the collar and the tube connection portion.

### FIELD OF TECHNOLOGY

The present invention relates to tubes and connectors for medical use, and particularly medical tube assemblies, medical tubes and connectors for use in breathing circuits suitable for delivering humidified gases to or from a patient, such as in respiratory humidification systems.

### BACKGROUND

In breathing circuits, various components transport warm and/or humidified gases to and from patients. Respiratory humidification helps reduce the likelihood of infection and/or tissue damage.

Humidifiers are used to provide humidification to the gases. Medical tubes are used to transport the humidified gases to and from a patient and to connect together any devices as part of a breathing circuit.

A breathing circuit may provide for a complete circuit of breathing gases to and from the patient. In some cases no tube is provided to remove gases from the patient and gas can be exhaled directly to atmosphere. In other cases a full breathing circuit including an inspiratory limb and an expiratory limb is provided to deliver gases to a patient as well as to remove them.

The breathing circuit may be used with a gases source for example a ventilator.

It is an object of the present invention to provide a medical tube assembly, and/or medical tube, and/or medical connector, or at least provide the public with a useful choice.

### SUMMARY

In an aspect of the disclosure there is provided a medical tube assembly, the medical tube assembly comprising:
a tube, the tube defining a gases passage extending from a first end of the tube to a second end of the tube, and wherein the tube comprises a water absorbing material, and
a connector defining a gases passage extending through the connector, the connector configured to connect to the first end of the tube,
wherein the tube comprises a main body portion and an increasing gases passage cross-sectional area portion, the increasing gases passage cross-sectional area portion at or near the first end of the tube and extending from the main body portion.

In some configurations, the connector comprises a tube connection portion provided inside the tube when the connector is connected to the tube.

In some configurations, the medical tube assembly comprises a collar, wherein the collar is configured to engage an external surface of the tube when the connector is connected to the tube.

In some configurations, the tube is retained between the collar and the tube connection portion.

In some configurations, a cuff region of the tube is retained between the collar and the tube connection portion.

In an aspect of the disclosure there is provided a medical tube assembly, the medical tube assembly comprising:
a tube, the tube defining a gases passage extending from a first end of the tube to a second end of the tube,
a connector defining a gases passage extending through the connector, the connector configured to connect to the first end of the tube,
wherein the connector comprises a tube connection portion configured to be provided inside the tube when the connector is connected to the tube, and
a collar, wherein the collar is configured to engage an external surface of the tube when the connector is connected to the tube, and
wherein the tube is retained between the collar and the tube connection portion, and
wherein the tube comprises a main body portion and an increasing gases passage cross-sectional area portion, the increasing gases passage cross-sectional area portion located at or near the first end of the tube and extending from the main body portion.

In some configurations, the tube comprises water absorbing material.

In some configurations, the tube is at least in part a corrugated tube.

In some configurations, the main body portion of the tube is corrugated.

In some configurations, corrugations comprise alternating crests and troughs.

In some configurations, the gases passage cross-sectional area of the main body portion of the tube is defined by:
a) a minimum cross-sectional area of the gases passage of the main body portion of the tube, or
b) a maximum cross-sectional area of the gases passage of the main body portion of the tube, or
c) when the tube is corrugated, a nominal cross-sectional area being an average of the minimum cross-sectional area of the gases passage of the main body portion of the tube and the maximum cross-sectional area of the gases passage of the main body portion of the tube.

In some configurations, the gases passage cross-sectional area of the main body portion of the tube is defined by:
a) a local minimum cross-sectional area of the gases passage of the main body portion of the tube, or
b) a local maximum cross-sectional area of the gases passage of the main body portion of the tube, or
c) when the tube is corrugated, a nominal cross-sectional area being an average of the minimum cross-sectional area of the gases passage of the main body portion of the tube and the maximum cross-sectional area of the gases passage of the main body portion of the tube.

In some configurations, one or more of: the local minimum cross-sectional area of the gases passage of the main body portion of the tube, the local minimum cross-sectional area of the gases passage of the main body portion of the tube and/or the local nominal cross sectional area of the gases passage of the main body portion of the tube is/are constant along a length of the main body portion of the tube.

In some configurations, the gases passage diameter of the main body portion of the tube is defined by:
a) a minimum diameter of the gases passage of the main body portion of the tube, or
b) a maximum diameter of the gases passage of the main body portion of the tube, or
c) when the tube is corrugated, a nominal cross-sectional diameter being an average of the minimum diameter of the gases passage of the main body portion of the tube and the maximum diameter of the gases passage of the main body portion of the tube.

In some configurations, the gases passage diameter of the main body portion of the tube is defined by:
a) a local minimum inner diameter of the gases passage of the main body portion of the tube, or
b) a local maximum inner diameter of the gases passage of the main body portion of the tube, or
c) when the tube is corrugated, a local nominal diameter of a gases passage diameter being an average of a diameter between opposing troughs of the tube and a diameter between opposing crests of the tube adjacent the trough.

In some configurations, one or more of: a the local minimum inner diameter of the gases passage of the main body portion of the tube, the local maximum inner diameter of the gases passage of the main body portion of the tube and/or the local nominal diameter of the gases passage of the main body portion of the tube is/are constant along a length of the main body portion of the tube.

In some configurations, the medical tube assembly comprises at least one heater wire.

In some configurations, the at least one heater wire is located in a wall of the tube, around the tube, or within the gases passage of the tube and/or the gases passage of the connector.

In some configurations, the medical tube assembly comprises an electrical connector configured to allow for electrical connection to the at least one heater wire.

In some configurations, the connector forms an elbow.

In some configurations, the connector is a straight connector.

In some configurations, the increasing gases passage cross-sectional area portion comprises an increase in a gases passage cross-sectional area in a direction towards the first end of the tube and/or towards a or the cuff region of the first end of the tube.

In some configurations, the increase in the gases passage cross-sectional area in the direction towards the first end of the tube and/or towards a or the cuff region of the first end of the tube is linear along a length of the increasing gases passage cross-sectional area portion.

In some configurations, the increasing gases passage cross-sectional area portion comprises a step change increase in gases passage cross-sectional area in a direction towards the first end of the tube and/or towards a or the cuff region of the first end of the tube.

In some configurations, the increasing gases passage cross-sectional area portion of the tube comprises an increasing cross-sectional area of the gases passage of the tube in a direction from the main body portion of the tube towards the first end of the tube.

In some configurations, the gases passage of the tube in the increasing gases passage cross-sectional area portion is defined by at least a diameter of the gases passage of tube in the increasing gases passage cross-sectional area portion.

In some configurations, the increasing gases passage cross-sectional area portion has an increasing diameter.

In some configurations, the increasing gases passage cross-sectional area portion comprises an increase in a gases passage diameter in a direction towards the first end of the tube and/or towards a or the cuff region of the first end of the tube.

In some configurations, the increase in the gases passage diameter in the direction towards the first end of the tube and/or towards a or the cuff region of the first end of the tube is linear along a length of the increasing gases passage cross-sectional area portion.

In some configurations, the increasing gases passage cross-sectional area portion comprises a step change increase in the gases passage diameter in the direction towards the first end of the tube and/or towards a or the cuff region of the first end of the tube.

In some configurations, the increasing gases passage cross-sectional area portion extends from a first location located away from the first end of the tube to a location near, or at the first end of the tube.

In some configurations, the increasing gases passage cross-sectional area portion extends from the main body portion towards the first end of the tube.

In some configurations, the increasing gases passage cross-sectional area portion extends from the main body portion towards a or the cuff region of the first end of the tube.

In some configurations, the increasing gases passage cross-sectional area portion of the tube is corrugated.

In some configurations, a cross-sectional area of the increasing gases passage cross-sectional area portion is defined by a local nominal cross-sectional area being an average of an area of the gases passage of the increasing gases passage cross-sectional area portion defined by a trough of the increasing gases passage cross-sectional area portion and an area of the gases passage of the increasing gases passage cross-sectional area portion defined by a crest of the increasing gases passage cross-sectional area portion adjacent the trough.

In some configurations, the cross-sectional area of the increasing gases passage cross-sectional area portion of the tube is defined by:
a) a local minimum cross-sectional area of the gases passage of the increasing gases passage cross-sectional area portion, or
b) a local maximum cross-sectional area of the gases passage of the increasing gases passage cross-sectional area portion.

In some configurations, the increasing gases passage cross-sectional area portion is defined by a number of corrugations.

In some configurations, the number of corrugations is about 1 to about 10 corrugations, or about 2 to about 7 corrugations, or about 2 to about 5 corrugations, or about 2 to about 2.5 corrugations, or about 2 corrugations.

In some configurations, when the tube is connected to the connector, an end of the increasing gases passage cross-sectional area portion is located at an end of the tube connection portion.

In some configurations, the increasing gases passage cross-sectional area portion is a flared portion.

In some configurations, the tube comprises a or the cuff region located at or near the first end of the tube.

In some configurations, the cuff region extends from the increasing gases passage cross-sectional area portion.

In some configurations, the cuff region extends towards a first end of the tube.

In some configurations, the cuff region of the first end of the tube is configured to engage with a or the tube connection portion of the connector.

In some configurations, the cuff region comprises at least one rib.

In some configurations, the cuff region comprises at least one rib orientated at least in part parallel to, or parallel to, a longitudinal axis of the tube.

In some configurations, the cuff region comprises at least one rib extending longitudinally along the tube.

In some configurations, the at least one rib is configured to engage with a retention feature of a or the collar of the medical tube assembly and/or the tube connection portion of the connector.

In some configurations, the cuff region has a larger inner cross-sectional area than a or the gases passage cross-sectional area of the main body portion of the tube.

In some configurations, the cuff region has a larger inner diameter than a or the gases passage diameter of the main body portion of the tube.

In some configurations, the cuff region is non-corrugated.

In some configurations, a wall of the tube is formed, at least in part, by a breathable material.

In some configurations, a wall of the tube is formed entirely by a breathable material.

In some configurations, a or the water absorbing material of the tube is a or the breathable material.

In some configurations, a or the collar is configured to engage an external surface of the connector when the connector is connected to the tube.

In some configurations, an internal surface of a or the collar comprises a retention feature, the retention feature configured to engage with an external surface of the tube to aid in retaining the tube on the connector.

In some configurations, the connector and/or the collar comprise at least one attachment feature, the attachment feature configured to retain the collar to the connector.

In some configurations, the at least one attachment feature comprises at least one attachment protrusion.

In some configurations, the at least one attachment feature comprises an attachment protrusion configured to engage a complementary recess.

In some configurations, the attachment protrusion is located on one of the connector or the collar, and the recess is located on the other of the connector or the collar.

In some configurations, a or the collar is configured to engage an external surface of a or the cuff region when the connector is connected to the tube.

In some configurations, a or the cuff region of the tube is retained between the collar and the tube connection portion.

In some configurations, a or the tube connection portion of the connector comprises a retention feature, the retention feature configured to engage with an internal surface of the tube to aid in retaining the tube on the connector.

In some configurations, the tube connection portion of the connector comprises a retention feature, the retention feature configured to engage an internal surface of a or the cuff region to aid in retaining the tube on the connector.

In some configurations, the retention feature comprises at least one retention protrusion.

In some configurations, the at least one retention protrusion is configured to extend at least in part circumferentially around the tube connection portion.

In some configurations, the at least one retention protrusion is configured to extend around an external surface of the tube connection portion.

In some configurations, the retention feature comprises a helical retention protrusion.

In some configurations, the tube connection portion and retention feature of a or the tube connection portion of the connector form a barb.

In some configurations, a or the tube connection portion and/or a or the collar comprise a sealing feature, the sealing feature configured to provide a pneumatic seal between the collar and tube connection portion.

In some configurations, the sealing feature comprises at least one sealing protrusion.

In some configurations, the at least one sealing protrusion is configured to extend at least in part circumferentially around the tube connection portion.

In some configurations, the at least one sealing protrusion is configured to extend around an external surface of the tube connection portion.

In some configurations, the sealing feature is located between a or the retention feature and a or the attachment feature.

In some configurations, the medical tube assembly comprises a second connector, the second connector connectable to the second end of the tube.

In some configurations, the second connector comprises any of the features of the connector as defined in any one of the preceding claims.

In some configurations, the tube comprises a second increasing gases passage cross-sectional area portion, the second increasing gases passage cross-sectional area portion is located at or near the second end of the tube and extending from the main body portion.

In some configurations, the second increasing gases passage cross-sectional area portion comprises an increase in a gases passage cross-sectional area in a direction towards the second end of the tube and/or towards a or the cuff region of the second end of the tube.

In some configurations, the increase in the gases passage cross-sectional area in the direction towards the second end of the tube and/or towards a or the cuff region of the second end of the tube is linear along a length of the second increasing gases passage cross-sectional area portion.

In some configurations, the second increasing gases passage cross-sectional area portion comprises a step change increase in the gases passage cross-sectional area in a direction towards the first end of the tube and/or towards a or the cuff region of the first end of the tube.

In some configurations, the second increasing gases passage cross-sectional area portion of the tube comprises an increasing cross-sectional area of the gases passage of the tube in a direction from the main body portion of the tube towards the second end of the tube.

In some configurations, the second increasing gases passage cross-sectional area is defined by at least a diameter of the gases passage in the second increasing gases passage cross-sectional area of the tube.

In some configurations, the increasing gases passage cross-sectional area portion has an increasing diameter.

In some configurations, the increasing gases passage cross-sectional area portion comprises an increase in a gases passage diameter in a direction towards the second end of the tube and/or towards a or the cuff region of the second end of the tube.

In some configurations, the increase in the gases passage diameter in the direction towards the second end of the tube and/or towards a or the cuff region of the second end of the tube linearly along a length of the second increasing gases passage cross-sectional area portion.

In some configurations, the second increasing gases passage cross-sectional area portion comprises a step change increase in the gases passage diameter in the direction towards the second end of the tube and/or towards a or the cuff region of the second end of the tube.

In some configurations, the second increasing gases passage cross-sectional area portion extends from a second location located away from the second end of the tube to a location near, or at the second end of the tube.

In some configurations, the second increasing gases passage cross-sectional area portion extends from the main body portion towards the second end of the tube.

In some configurations, the second increasing gases passage cross-sectional area portion extends from the main body portion towards a or the cuff region of the second end of the tube.

In some configurations, the second increasing gases passage cross-sectional area portion of the tube is corrugated.

In some configurations, a cross-sectional area of the second increasing gases passage cross-sectional area portion is defined by a local nominal cross-sectional area being an average of an area of the gases passage of the second increasing gases passage cross-sectional area portion defined by a trough of the second increasing gases passage cross-sectional area portion and an area of the gases passage of the second increasing gases passage cross-sectional area portion defined by a crest of the second increasing gases passage cross-sectional area portion adjacent the trough.

In some configurations, the increasing gases passage cross-sectional area portion of the tube is defined by:
a) a local minimum cross-sectional area of the gases passage of the increasing gases passage cross-sectional area portion, or
b) a local maximum cross-sectional area of the gases passage of the increasing gases passage cross-sectional area portion

In some configurations, the second increasing gases passage cross-sectional area portion is defined by a number of corrugations.

In some configurations, the number of corrugations is about 1 to about 10 corrugations, or about 2 to about 7 corrugations, or about 2 to about 5 corrugations, or about 2 to about 2.5 corrugations, or about 2 corrugations.

In some configurations, when the tube is connected to the second connector an end of the second increasing gases passage cross-sectional area portion is located at or near the tube connection portion of the second connector.

In some configurations, the second increasing gases passage cross-sectional area portion is a flared portion.

In some configurations, the tube comprises a second cuff region at or near the second end of the tube.

In some configurations, the cuff region extends from a or the second increasing gases passage cross-sectional area portion.

In some configurations, the cuff region extends towards a second end of the tube.

In some configurations, the cuff region of the second end of the tube is configured to engage with the tube connection portion of the connector.

In some configurations, the cuff region of the second end of the tube comprises at least one rib.

In some configurations, the cuff region of the second end of the tube comprises at least one rib orientated at least in part parallel to, or parallel to, a longitudinal axis of the tube.

In some configurations, the cuff region comprises at least one rib extending longitudinally along the tube.

In some configurations, the at least one rib is configured to engage with a retention feature of the collar and/or the tube connection portion of the connector.

In some configurations, the cuff region of the second end of the tube has a larger inner cross-sectional area than a or the gases passage cross-sectional area of the main body portion of the tube.

In some configurations, the cuff region has a larger inner diameter than a or the gases passage diameter of the main body portion of the tube.

In some configurations, the cuff region is non-corrugated.

In an aspect of the disclosure there is provided a expiratory pathway as an expiratory tube, a filter and a Y-piece, wherein the expiratory tube is the medical tube assembly of any of the above claims.

In some configurations, a pressure drop across the expiratory pathway is less than 1.8 cmH2O for a flow of gases at 30 Litres/minute, or 1.8 cmH2O at 15 Litres/minute, or 1.85 cmH2O at 2.5 Litres/minute.

In an aspect of the disclosure there is provided a breathing circuit comprising: the medical tube assembly of any of the above aspects as an expiratory tube, a dryline configured to extend from a gases source, a humidification chamber or humidifier configured to connect to the dry line, and an inspiratory tube configured to connect to the humidifier chamber or humidifier to a Y-piece.

In some configurations, a pneumatic compliance of the breathing circuit is less than 5 ml/cmH2O at 60 cmH2O.

In an aspect of the disclosure there is provided a medical tube assembly, the medical tube assembly comprising:
a tube, the tube defining a gases passage extending from a first end of the tube to a second end of the tube, and wherein the tube comprises:
   a main body portion defining a main body portion gases passage,
   a first increasing gases passage cross-sectional area portion located at or near the first end of the tube and extending from the main body portion, the first increasing gases passage cross-sectional area portion increasing in gases passage cross-sectional area in a direction towards the first end of the tube to a cross-sectional area of a gases passage of an end of the first increasing gases passage cross-sectional area portion, and
   a second increasing gases passage cross-sectional area portion located at or near the second end of the tube and extending from the main body portion, the second increasing gases passage cross-sectional area portion increasing in gases passage cross-sectional area in a direction towards the second end of the tube to a cross-sectional area of a gases passage of an end of the second increasing gases passage cross-sectional area portion,
a first connector configured to connect to the first end of the tube, the first connector defining a gases passage extending through the first connector, the first connector comprising:
   a tube connection portion configured to engage with the first end of the tube, the tube connection portion defining a gases passage,
   a second portion defining a gases passage,
   and wherein,
   a) the cross-sectional area of the gases passage of the end of the first increasing gases passage cross-sectional area portion is greater than a minimum cross-sectional area of the tube connection portion gases passage of the first connector,
   b) a minimum cross-sectional area of the gases passage of the second portion of the first connector is greater than the minimum cross-sectional area of the gases passage of the tube connection portion of the first connector, and
a second connector being configured to connect to the second end of the tube, the second connector defining a gases passage extending through the second connector, the second connector comprising:
   a tube connection portion configured to engage with the second end of the tube, the tube connection portion defining a gases passage,
   a second portion defining a gases passage,
   and wherein,
   a) the cross-sectional area of the gases passage of end of the second increasing gases passage cross-sectional area portion is greater than a minimum cross-sectional area of the gases portion of the tube connection portion of the second connector,
   b) the minimum cross-sectional area of the gases passage of second portion of the second connector is greater than a minimum cross-sectional area of the gases passage of the tube connection portion of second connector.

In some configurations, the second portion of the first connector is an associated connector portion configured to engage with a first associated connector.

In some configurations, the second portion of the first connector is an intermediate portion located between the tube connection portion of the first connector and an associated connector portion configured to engage with a first associated connector.

In some configurations, the first associated connector comprises a taper configured to engage with the associated connector portion of the first connector.

In some configurations, the second portion of the second connector is an associated connector portion configured to engage with a second associated connector.

In some configurations, the second portion of the second connector is an intermediate portion located between the tube connection portion of the second connector and an associated connector portion configured to engage with a second associated connector.

In some configurations, the second associated connector comprises a taper configured to engage with the associated connector portion of the second connector.

In some configurations, a cross-sectional area of a gases passage of an opening of the second associated connector is less than a minimum cross-sectional area of a gases passage of the medical tube assembly.

In some configurations, the tube comprises a cuff region located at or near the first end of the tube and/or a cuff region located at or near the second end of the tube.

In some configurations, the cuff region of the first end of the tube is retained between a first collar and the tube connection portion of the first connector and/or wherein the cuff region of the second end of the tube is retained between a second collar of the second portion and the tube connection portion.

In some configurations, the tube comprises water absorbing material.

In an aspect of the disclosure there is provided a medical tube assembly, the medical tube assembly comprising:
a tube, the tube comprising:
   a main body portion,
   a first increasing gases passage cross-sectional area portion located at or near the first end of the tube and extending from the main body portion, and
   a second increasing gases passage cross-sectional area portion located at or near the second end of the tube and extending from the main body portion,
   a first connector having a tube connection portion for connection to the tube at a first end of the tube and a second portion,
   a second connector having a tube connection portion for connection to the tube at a second end of the tube and a second portion,
   wherein at least the tube connection portion, the second portion of the first connector, tube, the tube connection portion and second portion of the second connector define a gases passage of the assembly,
   wherein in a direction towards the first end of the tube, the gases passage:
   a) increases in gases passage cross-sectional area from an end of the main body portion nearest the first increasing gases passage cross-sectional area portion through the first increasing gases passage cross-sectional area portion,
   b) decreases in gases passage cross-sectional area from the first increasing gases passage cross-sectional area portion to the tube connection portion of the first connector, and
   c) increases in gases passage cross-sectional area from the tube connection portion of the first connector to the second portion of the first connector, and,
   wherein in a direction towards the second end of the tube, the gases passage:
   a) increases in gases passage cross-sectional area from an end of the main body portion nearest the second increasing gases passage cross-sectional area portion through the second increasing gases passage cross-sectional area portion,
   b) decreases in gases passage cross-sectional area from the second increasing gases passage cross-sectional area portion to the tube connection portion of the second connector, and
   c) increases in gases passage cross-sectional area from the tube connection portion of the second connector to the second portion of the second connector.

In some configurations, the second portion of the first connector is an associated connector portion configured to engage with a first associated connector.

In some configurations, the second portion of the first connector is an intermediate portion located between the tube connection portion of the first connector and an associated connector portion configured to engage with a first associated connector.

In some configurations, the first associated connector comprises a taper configured to engage with the associated connector portion of the first connector.

In some configurations, a cross-sectional area of a gases passage of an opening of the first associated connector is less than a minimum cross-sectional area of the gases passage of the medical tube assembly.

In some configurations, the second portion of the second connector is an associated connector portion configured to engage with a second associated connector.

In some configurations, the second portion of the second connector is an intermediate portion located between the tube connection portion of the second connector and an associated connector portion configured to engage with a second associated connector.

In some configurations, the second associated connector comprises a taper configured to engage with the associated connector portion of the second connector.

In some configurations, a cross-sectional area of a gases passage of an opening of the second associated connector is less than a minimum cross-sectional area of the gases passage of the medical tube assembly.

In some configurations, the tube comprises a cuff region located at or near the first end of the tube and/or a cuff region located at or near the second end of the tube.

In some configurations, the cuff region of the first end of the tube is retained between a first collar and the tube connection portion of the first connector and/or wherein the cuff region of the second end of the tube is retained between a second collar of the second portion and the tube connection portion.

In some configurations, the tube comprises water absorbing material.

In some configurations, the first and/or second increasing gases passage cross-sectional area portion of the tube is/are corrugated.

In some configurations, a cross-sectional area of the first increasing gases passage cross-sectional area portion is defined by a local nominal cross-sectional area being an average of an area of the gases passage of the first increasing gases passage cross-sectional area portion defined by a trough of the first increasing gases passage cross-sectional area portion and an area of the gases passage of the first increasing gases passage cross-sectional area portion defined by a crest of the first increasing gases passage cross-sectional area portion adjacent the trough.

In some configurations, a cross-sectional area of the second increasing gases passage cross-sectional area portion is defined by a local nominal cross-sectional area being an average of an area of the gases passage of the second increasing gases passage cross-sectional area portion defined by a trough of the second increasing gases passage cross-sectional area portion and an area of the gases passage of the second increasing gases passage cross-sectional area portion defined by a crest of the second increasing gases passage cross-sectional area portion adjacent the trough.

In some configurations, the first increasing gases passage cross-sectional area portion of the tube is defined by:
a) a local minimum cross-sectional area of the gases passage of the increasing gases passage cross-sectional area portion, or
b) a local maximum cross-sectional area of the gases passage of the increasing gases passage cross-sectional area portion.

In some configurations, the second increasing gases passage cross-sectional area portion of the tube is defined by:
a) a local minimum cross-sectional area of the gases passage of the increasing gases passage cross-sectional area portion, or
b) a local maximum cross-sectional area of the gases passage along the length of the increasing gases passage cross-sectional area portion.

In some configurations, a cross sectional diameter of the main body portion of the tube is less than a cross sectional diameter of the first increasing gases passage cross-sectional area portion and/or the first increasing gases passage cross-sectional area portion.

In an aspect of the disclosure there is provided a medical tube assembly, the medical tube assembly comprising:
a tube, the tube defining a gases passage extending from a first end of the tube to a second end of the tube,
a connector being configured to connect to the first end of the tube, the connector defining a gases passage extending through the connector, and
wherein the connector is configured to connect to an associated connector, the associated connector comprising an opening configured to convey gases to and/or receive gases from the gases passage of the connector when the associated connector is connected to the connector, and
wherein
   a) a cross-sectional area of the gases passage of the main body portion of the tube, and
   b) a minimum cross-sectional area of the gases passage of the connector,
   are both equal to or greater than a cross-sectional area of a gases passage of the opening of the associated connector, and
wherein the tube comprises an increasing gases passage cross-sectional area portion located near the first end of the tube.

In an aspect of the disclosure there is provided a medical tube assembly, the medical tube assembly comprising:
a tube, the tube defining a gases passage extending from a first end of the tube to a second end of the tube,
a first connector being configured to connect to the first end of the tube, the first connector defining a gases passage extending through the first connector,
a second connector being configured to connect to the second end of the tube, the second connector defining a gases passage extending through the second connector, and
wherein the first connector is configured to connect to an associated connector, the associated connector comprising an opening configured to receive gases from or convey gases to the gases passage of the connector when the associated connector is connected to the connector, and
wherein:
a) a cross-sectional area of the gases passage of the main body portion of the tube,
b) a minimum cross-sectional area of the gases passage of the first connector, and
c) a minimum cross-sectional area of the gases passage of the second connector,
are all equal to or greater than a cross-sectional area of the opening of the associated connector.

In some configurations, the second connector is configured to connect to a second associated connector, the second associated connector comprising an opening configured to receive gases from or convey gases to the gases passage of the connector when the second associated connector is connected to the second connector, and wherein:
a) a cross-sectional area of the gases passage of the main body portion of the tube,
b) a minimum cross-sectional area of the gases passage of the first, connector, and
c) a minimum cross-sectional area of the gases passage of the second connector,
are all equal to or greater than a cross-sectional area of the opening of the second associated connector

In some configurations, the gases passage cross-sectional area of the main body portion of the tube is defined by:
a) a minimum cross-sectional area of the gases passage of the main body portion of the tube, or
b) a maximum cross-sectional area of the gases passage of the main body portion of the tube, or
c) when the tube is corrugated, a nominal cross-sectional area being an average of the minimum cross-sectional area of the gases passage of the main body portion of the tube and the maximum cross-sectional area of the gases passage of the main body portion of the tube.

In some configurations, the gases passage cross-sectional area of the main body portion of the tube is defined by:
a) a local minimum cross-sectional area of the gases passage of the main body portion of the tube, or
b) a local maximum cross-sectional area of the gases passage of the main body portion of the tube, or
c) when the tube is corrugated, a nominal cross-sectional area being an average of the minimum cross-sectional area of the gases passage of the main body portion of the tube and the maximum cross-sectional area of the gases passage of the main body portion of the tube.

In some configurations, one or more of: the local minimum cross-sectional area of the gases passage of the main body portion of the tube, the local minimum cross-sectional area of the gases passage of the main body portion of the tube and/or the local nominal cross sectional area of the gases passage of the main body portion of the tube is/are constant along a length of the main body portion of the tube.

In some configurations, the gases passage diameter of the main body portion of the tube is defined by:
a) a minimum diameter of the gases passage of the main body portion of the tube, or
b) a maximum diameter of the gases passage of the main body portion of the tube, or
c) when the tube is corrugated, a nominal cross-sectional diameter being an average of the minimum diameter of the gases passage of the main body portion of the tube and the maximum diameter of the gases passage of the main body portion of the tube.

In some configurations, the gases passage diameter of the main body portion of the tube is defined by:
a) a local minimum inner diameter of the gases passage of the main body portion of the tube, or
b) a local maximum inner diameter of the gases passage of the main body portion of the tube, or
c) when the tube is corrugated, a local nominal diameter of a gases passage diameter being an average of a diameter between opposing troughs of the tube and a diameter between opposing crests of the tube adjacent the trough.

In some configurations, one or more of: the local minimum inner diameter of the gases passage of the main body portion of the tube, the local maximum inner diameter of the gases passage of the main body portion of the tube and/or the local nominal diameter of the gases passage of the main body portion of the tube is/are constant along a length of the main body portion of the tube.

In an aspect of the disclosure there is provided a medical tube assembly, the medical tube assembly comprising:
a tube, the tube defining a gases passage extending from a first end of the tube to a second end of the tube,
a connector being configured to connect to the first end of the tube, the connector defining a gases passage extending through the connector, and
wherein the connector is configured to connect to an associated connector, the associated connector comprising an opening configured to receive gases from, or convey gases to the gases passage of the connector when the associated connector is connected to the connector,
a first tube diameter defined by a main body portion of the tube,
a second tube diameter defined by the tube at the first end of the tube,
a tube end connector diameter defined by the minimum diameter of a gases passage of a tube connection portion of the connector,
wherein the second tube diameter is greater than the first tube diameter and the tube end connector diameter, and
wherein the first tube diameter, second tube diameter and tube end connector diameter are all greater than a diameter of the opening of the associated connector.

In an aspect of the disclosure there is provided a breathing kit comprising an inspiratory tube, an expiratory tube as defined by any of the above aspects, and a humidification chamber.

In an aspect of the disclosure there is provided a breathing kit comprising an inspiratory tube, an expiratory tube as defined by any of the above aspects, a humidification chamber and a y-piece.

In an aspect of the disclosure there is provided a breathing kit comprising an inspiratory tube, an expiratory tube as defined by any of the above aspects, a humidification chamber, a y-piece and a dry line.

In an aspect of the disclosure there is provided a breathing kit comprising an inspiratory tube, an expiratory tube as defined by any of the above aspects, a humidification chamber, a y-piece, a dry line and a filter.

It will be appreciated that any of the above aspects may be combined with any combination of the above aspects.

The term “axis” as used in this specification means the axis of revolution about which a line or a plane may be revolved to form a symmetrical shape. For example, a line revolved around an axis of revolution will form a surface, while a plane revolved around an axis of revolution will form a solid.

As used herein the term “and/or” means “and” or “or”, or both.

As used herein “(s)” following a noun means the plural and/or singular forms of the noun.

For the purpose of this specification, where method steps are described in sequence, the sequence does not necessarily mean that the steps are to be chronologically ordered in that sequence, unless there is no other logical manner of interpreting the sequence.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying drawings.

### BRIEF DESCRIPTION OF THE DRAWINGS

Embodiments of the invention will be described by way of example only and with reference to the drawings, in which:

FIG. 1 shows a schematic view of a respiratory assistance apparatus configured to provide a respiratory therapy to a patient.

FIGS. 2 and 3 show a schematic view of another embodiment of a respiratory assistance apparatus configured to provide a respiratory therapy to a patient.

FIG. 4 shows a perspective view of an embodiment of the medical tube assembly.

FIG. 5 shows a side view of an embodiment of the medical tube assembly.

FIG. 6 shows an exploded side view of an embodiment of the medical tube assembly.

FIG. 7 shows a cross-sectional side view of an embodiment of the medical tube assembly.

FIG. 8 shows a cross-sectional side view of an embodiment of the medical tube assembly.

FIG. 9 shows an exploded cross-sectional side view of the first connector (for example a device end connector) and the tube of an embodiment of the medical tube assembly.

FIG. 10 shows a cross-sectional side view of the first connector (for example a device end connector) and the tube of an embodiment of the medical tube assembly.

FIG. 11 shows a cross-sectional side view of the first connector (for example a device end connector) and the tube of an embodiment of the medical tube assembly; in particular, it shows a heater wire and an electrical connector.

FIG. 12 shows an exploded cross-sectional side view of the second connector (for example a patient end) and the tube of the medical tube assembly.

FIG. 13 shows a cross-sectional side view of the first connector (for example a device end) and the tube of the medical tube assembly.

FIG. 14 shows a cross-sectional side view of the medical tube assembly.

FIG. 15 shows a perspective view of the first connector (for example a device end) and the tube of the medical tube assembly; in particular, it shows the location of the cross-sections shown in proceeding FIGS. 16-19.

FIG. 16 shows a perspective view of a cross-section of a main body portion of a tube of the medical tube assembly.

FIG. 17 shows a perspective view of a cross-section of an increasing gases passage cross-sectional area portion of the tube.

FIG. 18 shows a perspective view of a cross-section of the medical tube assembly, including a tube connection portion of a connector of the medical tube assembly.

FIG. 19 shows a perspective view of a cross-section of the medical tube assembly, including a cuff region of the tube.

FIGS. 20-22 show cross-sections of the tube illustrating different cross-sectional areas of the gases passage of the increasing gases passage cross-sectional area portion and the main body portion of the tube.

FIG. 23 shows a cross-sectional side view of the second connector (for example a patient end) and the tube of the medical tube assembly; in particular, it shows the relative dimensions of the tube connection portion and an associated connector.

FIG. 24 shows a cross-sectional side view of the second connector (for example a patient end) and the tube of the medical tube assembly; in particular, it shows the relative dimensions of the tube and the associated connector.

### DETAILED DESCRIPTION OF PREFERRED EMBODIMENTS

Reference is made in detail to an embodiment of the present invention, examples of which is illustrated in the accompanying drawings.

Medical tubes can be used in breathing circuits or respiratory systems, for example, for delivering and/or removing humidified gases from a patient, such as in obstructive sleep apnea, respiratory humidification, and surgical humidification systems including insufflation systems and systems for patients undergoing procedures under general anesthetic.

This application relates to medical tube assemblies, medical tubes and connectors for use in breathing circuits or respiratory systems. The medical tubes and connectors may be used for delivering and/or removing humidified gases from a patient, such as in obstructive sleep apnea, respiratory humidification, and surgical humidification systems including insufflation systems and systems for patients undergoing procedures under general anesthetic.

The medical tube assemblies can be used to deliver gases between two components of a breathing circuit. Medical tube assemblies can be used to deliver gases between a component and a patient. For example, between a humidifier and a patient.

In some embodiments, the medical tubes can be inspiratory tubes, expiratory tubes, patient interface tubes, supply tubes, dry lines, insufflation tubes, etc.

The medical tube assemblies may comprise one or more medical tubes, and one or more connectors.

Tubes for use in a respiratory device may be designed to reduce resistance to flow for example see: U.S. patent application Ser. No. 16/317,920 published as U.S. Patent Application Publication No. 20210252248 and the National Phase Entry of PCT Publication No. WO2018/016975, U.S. patent application Ser. No. 13/698,272 published as U.S. Patent Application Publication No. 20130112201 and the National Phase Entry of PCT Publication No. WO2011/149362, U.S. patent application Ser. No. 16/970,510 published as U.S. Patent Application Publication No. 20210077765 and the National Phase Entry of PCT publication No. WO2019/164409 U.S. patent application Ser. No. 16/964,411 published as U.S. Patent Application Publication No. 20210046272 and the National Phase Entry of PCT Publication No WO 2019/147142 all herein incorporated by reference.

This application claims priority from U.S. provisional patent application 63/366,611 filed 17 Jun. 2022, the entire contents of which is hereby incorporated by reference in its entirety.

The medical tubes, connectors and medical tube assemblies described herein can be provided in one or more respiratory systems, breathing circuits, or kits.

Pneumatic compliance of a breathing circuit may be a design consideration for tubes and tube assemblies as part of a breathing circuit. Pneumatic compliance may be measured by a change in volume of a tube, medical tube assembly and/or breathing circuit for a given pressure change. Pneumatic compliance may have particular importance when low tidal volumes of gases are being provided to the patient (for example in a neonatal and/or paediatric setting). The lower the compressible volume of a breathing circuit, the lower the pneumatic compliance of the breathing circuit for a fixed stiffness. Accuracy in tidal volume delivery may be important in ensuring that adequate therapy is provided to a patient, for example the lower the pneumatic compliance of a breathing circuit relative to the patient lung compliance, the less potential there is for error in delivered tidal volume. Overall pneumatic compliance of the breathing circuit may for example be based on the initial volume of the breathing circuit and stiffness of the various components (a low stiffness may cause the volume to increase).

Another design consideration is resistance to flow for the breathing circuit.

Resistance to flow may be measured by the gases pressure drop for a given flow rate. The higher the resistance to flow in the breathing circuit the more difficult it will be for a gases source to provide a given flow rate and/or for the patient to exhale. Decreasing resistance to flow may be accomplished by for example increasing the diameter of the tube, or connectors, however this will also increase pneumatic compliance (by increasing the overall compressible volume of the circuit) as described above, which may be undesirable. There may therefore be a trade-off between keeping both pneumatic compliance and resistance to flow desirably low. It may be particularly desirable to decrease resistance to flow for the circuit (for example by decreasing resistance to flow for a tube of the circuit) while not having a significant impact on the pneumatic compliance of the circuit.

The disclosure sets out various tubes and tube assemblies which may decrease overall resistance to flow, while not having a significant the impact on pneumatic compliance.

One example of a way of reducing resistance to flow of the medical tube assembly is by increasing a cross-sectional area of a gases passage of a connector 100 (as described in more detail below). However, this may be constrained by the diameter of the tube, and the connection type (for example a barbed type connection with a collar) as the gases passage of the connector may need to be provided inside the tube, which can introduce a restriction to flow. One way to approach this would be to increase the cross-sectional area of the gases passage tube itself, however, as described above this would increase compressible volume and therefore pneumatic compliance of the assembly. An increasing gases passage cross-sectional area of the tube (as described in more detail below) provided at or near the end of the tube may allow for an increase of the cross-sectional area of the gases passage of a connector 100 while still providing a barbed type connection with a collar. This may provide for a relatively small increase in compressible volume of the medical tube assembly (and therefore pneumatic compliance) but a relatively large decrease in resistance to flow of the medical tube assembly, and an overall better performing tube.

The medical tubes may be used for delivering and/or removing humidified gases from a patient, such as in obstructive sleep apnea, respiratory humidification, and surgical humidification systems including insufflation systems and systems for patients undergoing procedures under general anesthetic. The medical conduits can be used to deliver respiratory gases to and/or from a patient as part of a respiratory therapy or treatment. The respiratory gases may be heated and/or humidified prior to delivery to the patient in order to, for example, reduce the likelihood of infection and/or tissue damage.

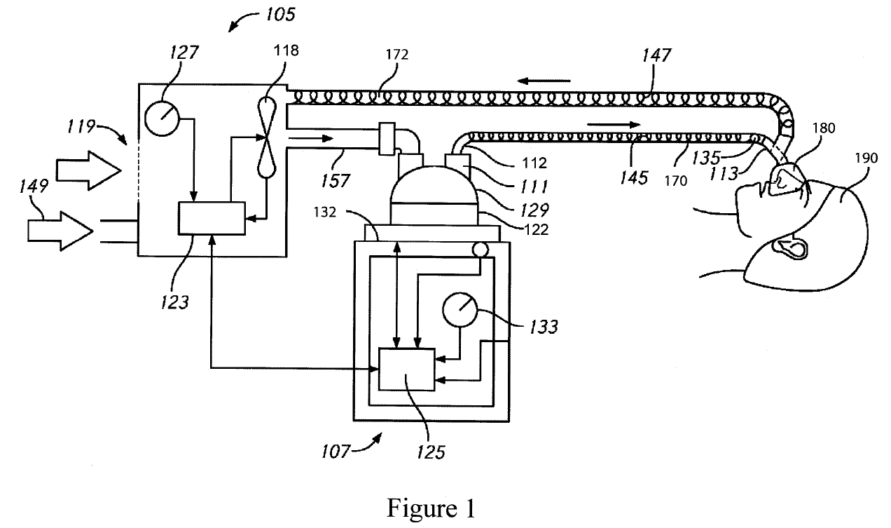

FIG. 1 schematically illustrates an example respiratory assistance apparatus including a breathing circuit comprising one or more tubes 170, 172, a patient interface 180 and a Y-piece 113. The respiratory assistance apparatus may be a ventilator, a continuous, variable, or bi-level positive airway pressure (PAP) system or other form of respiratory therapy, such as, for example, high flow therapy.

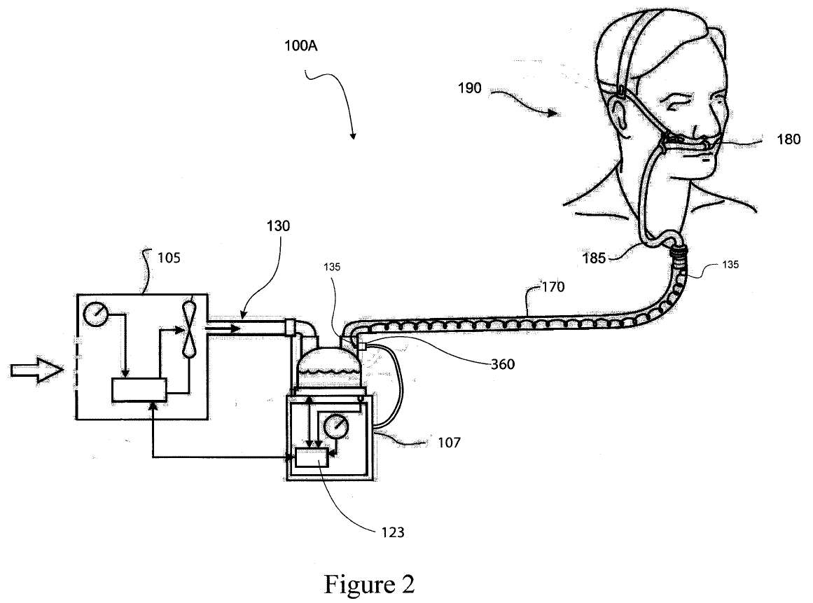

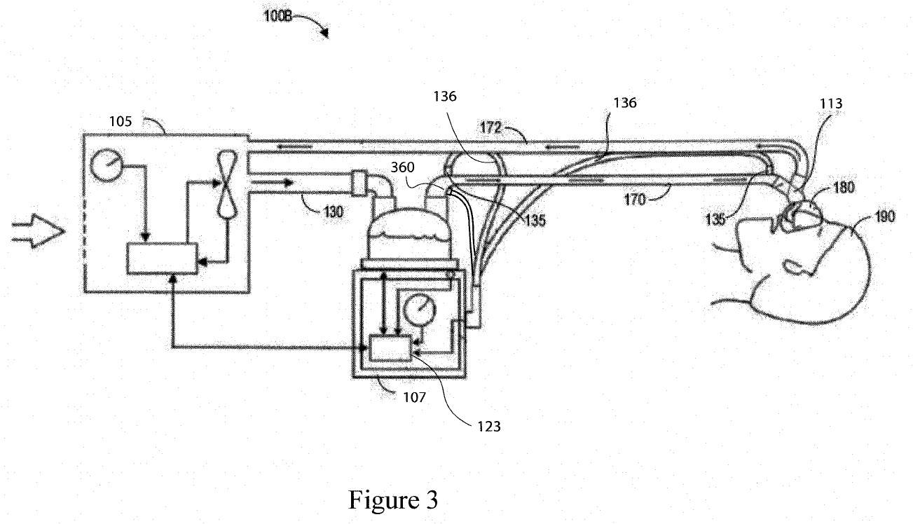

Gases may be transported in the breathing circuit of FIG. 1 as follows. Dry or relatively dry gases pass from a gases source 105 through a supply tube 157 (or interchangeable a dry line 130 for example as shown in FIGS. 2 and 3) to a humidifier 107, which humidifies the dry gases. The gases source 105 may be, for example, a ventilator or a blower. The gases source 105 may draw air from the ambient environment and/or from a gases reservoir (for example an oxygen source such as an oxygen bottle) as described in more detail below. The humidifier 107 connects to an end 109 of the inspiratory tube 170, via a port 112. The inspiratory tube 170 is connected to a patient 190 through a patient interface 180, optionally using a Y-piece 113. An optional expiratory tube, such as expiratory tube 172, also connects to the patient interface 180 through the Y-piece 113. The expiratory tube 172 may be configured to move exhaled gases away from the patient 190. As illustrated in FIG. 1, expiratory tube 172 returns exhaled gases from the patient 190 to the gases source 105. Alternatively, the inspiratory tube 170 connects directly to the patient interface 180 without a Y-piece 113. In such an implementation, expired gases are allowed to flow directly to the ambient environment.

Inspiratory tube 170 can include electrically conductive elements such as heater and/or sensor elements 145. Similarly, expiratory tube 172 can include heater and/or sensor elements 147. Further, the Y-piece 113 and patient interface 180 can also include heater and/or sensor elements. As will be explained in further detail below, the heater and/or sensor elements 145, 147 can be wires or filaments.

The heater and/or sensor elements 145 may be located in a wall of the tube, around the tube (for example wrapped around an external surface of the tube), or within a gases passage of the tube.

In some configurations the heater and/or sensor elements 145 may connect to a sensor(s) 135 to communicate any outputs from the sensor to a controller 123.

The heater and/or sensor elements 145 may comprise separate heating elements and/or sensor elements. For example, the heater and/or sensor elements 145 may comprise a pair of heating elements and a pair of sensor elements.

In some configurations, the heater and/or sensor elements 145 comprise only heating elements and the sensor(s) 135 is electrically connected to the controller by a lead 136 for example as shown in FIG. 3.

As shown in the example respiratory assistance apparatus of FIG. 1, gases (for example dry or relatively dry gases relative to the humidified gases delivered to the user) enter the gases source 105 through a vent 119. A fan 118 may improve gas flow into the gases source 105 by drawing air or other gases through the vent 119. The fan 118 may be, for instance, a variable speed fan, where an electronic controller 123 controls the fan speed. The electronic controller 123 may also be controlled by a second electronic controller 125, or vice versa, in some implementations.

The humidifier 107 can include a humidification chamber 129 containing a volume of water 122 or other suitable humidifying liquid. The humidification chamber 129 can be removable from the humidifier 107. The humidification chamber 129 may include a highly heat-conductive base (for example, an aluminum base) contacting or associated with a heater plate 132 on the humidifier 107.

The humidifier 107 may also include electronic controls. In FIG. 1, for example, the humidifier 107 includes an electronic, analog, or digital controller 125. The controller 125 may be a microprocessor-based controller executing computer software commands stored in associated memory. In response to humidity, temperature or other feedback values provided via a user interface 133 and/or integrated sensors, the controller 125 determines heat, flow, pressure and/or other variables used to provide humidified gases to a patient (also referred to herein as a user). User interface 133 can be one or more hardware buttons and/or a display or touch screen display. The display can provide audio and/or visual feedback to the user. When condensation is detected, any number of alarms, alerts, feedback, guidance or instructions can be provided to the user to indicate the presence, extent or remedies for a condensation condition. For example, the user interface can provide an alarm when condensation is detected. The user interface can provide a visual indication of condensation. The user interface can also provide an animation to instruct a user how to properly drain condensation.

Any suitable patient interface may be used. Patient interface is a broad term and is to be given its ordinary and customary meaning to a person of ordinary skill in the art (that is, it is not to be limited to a special or customized meaning) and includes, without limitation, masks (such as tracheal mask, face masks, and nasal masks), endotracheal tubes, tracheostomy tubes, cannulas, and nasal pillows. A sensor(s) 135 (for example a temperature probe) may be incorporated in or connected to inspiratory tube 170 near the Y-piece 113, or directly to the Y-piece 113 or the patient interface 180. The temperature probe 135 monitors the temperature of the flow of gases near or at the patient interface 180. A heating wire (such as element 145) may be used to adjust the temperature of the patient interface 180, the Y-piece 113, and/or the inspiratory tube 170 to maintain the temperature of the flow of gases above the saturation temperature, thereby reducing the opportunity for unwanted condensation, and/or to deliver the gases at optimal temperature for patient therapy (for example, 40° C. at the patient end of the inspiratory tube and/or 37° C. at the patient for non-invasive therapy). As shown in FIG. 1, exhaled gases are optionally returned from the patient interface 180 to the gases source 105 via the expiratory tube 172.

In some configurations, the sensor(s) 135 may comprise a thermistor. In examples where the sensor(s) is a temperature probe the thermistor may be part of the temperature probe.

In some embodiments, an additional conduit, such as an interface conduit 185, can connect between the inspiratory tube 170 and the patient interface 180. It is to be understood that other variations from the system shown may exist. For example, the inspiratory tube 170 may comprise multiple sections to accommodate other equipment such as a water trap, an intermediate connector and optionally one or more sensors, a PCB, and/or a controller.

The system of FIG. 1 may be readily adapted for other applications involving the supply of a heated and/or humidified gas flow to a user or patient, including but not limited to laparoscopy, and the like. Such applications may use alternative gases, operating parameters (e.g., flow, pressure, temperature, or humidity) and patient interfaces. Further, although shown with respect to a separate ventilator and humidifier system, it is to be understood that the present disclosure can also be used with an integrated ventilator/blower and humidifier system. For example, the gases source 105 may be integrated with, or a separate component from, the humidifier 107.

The system of FIG. 1 can also provide oxygen (O2) or an O2 fraction to the user through port 149. The system of FIG. 1 can receive O2 from a remote source and/or by blending atmospheric air with incoming O2 from the remote source. The blending of atmospheric air and incoming O2 can occur via a Venturi or a similar inlet located in gases source 105 or humidifier 107.

FIGS. 2 and 3 schematically illustrate embodiments of a respiratory assistance apparatus 100A and 100B that can include one or more of the medical tubes described herein. In the illustrated embodiment of FIGS. 2 and 3, the respiratory system 100A, 100B includes a gases source 105 that is either integrated with, or a separate component from the humidifier 107.

FIG. 3 shows an example system comprising an expiratory tube 172 and an inspiratory tube 170.

The gases source 105 and humidifier 107 supply heated and humidified gases to a patient 190 via a breathing circuit that includes, for example, an inspiratory tube 170 and a patient interface 180 as illustrated for example in FIGS. 1-3.

For example, as described above, a sensor(s) 135 can connect to the inspiratory tube 170 near the patient interface 180 or a sensor(s) 135 can connect to the patient interface 180, among other possible sensor locations. The sensor(s) 135 can be integrated into or connectable to the inspiratory tube 170. In some configurations, the system includes two sensors 135, with a first sensor 135 positioned at or nearby to the humidifier chamber outlet end of the inspiratory tube 170, and a second sensor 135 positioned at the patient end of the inspiratory tube 170 (for example as shown in FIG. 3).

As described above in some configurations for example as shown in FIG. 3, the sensor(s) 135 may be electrically connected to the apparatus controller by a lead 136.

In some configurations, the sensor(s) 135 can be connected to the sensor port 360 of the connector and/or a sensor port of the inspiratory tube (or a connector of the inspiratory tube). Alternatively, or in addition, sensor(s) 135 may be provided at or nearby to the humidifier chamber inlet. A signal provided by the sensor(s) 135 can be provided, for example, to a control system (for example controller 123 as shown in FIG. 1). In some embodiments, the sensor(s) 135 comprises one or more of a temperature sensor, a humidity sensor, a flow sensor, and a pressure sensor. Although, in FIG. 3 the sensors 135 are illustrated connected to the patient end and chamber outlet end of the inspiratory tube 170, one or more sensors can be included, alternatively or additionally, in other locations on the inspiratory tube 170 and/or on other medical tubes or components in the respiratory system.

It will be appreciated that the sensor arrangement as described with respect to FIG. 2 may be applied to the system of FIG. 1.

It is to be understood that other variations from the system shown may exist. For example, the inspiratory 170 and/or expiratory tube 172 may comprise multiple sections to optionally accommodate other equipment such as a water trap, an intermediate connector with one or more sensors, a PCB, and/or a controller. In another example embodiment, the system may include a nebulizer or a port therefore. In another example embodiment, the system may include a catheter mount or an exhalation valve (for example a PEEP valve).

The medical tube assemblies, medical tubes and/or connectors may also be provided as a breathing kit.

In some configurations, the breathing kit may comprise an inspiratory tube 170, an expiratory tube 172, and a humidification chamber 129.

In some configurations, the breathing kit may comprise an inspiratory tube 170, an expiratory tube 172, a humidification chamber 129 and a y-piece 113.

In some configurations, the breathing kit may comprise an inspiratory tube 170, an expiratory tube 172, a humidification chamber 129, a y-piece 113 and a dry line 130.

In some configurations, the breathing kit may comprise an inspiratory tube 170, an expiratory tube 172, a humidification chamber 129, a y-piece 113, a dry line 130 and a filter.

The breathing kit may also comprise at least one of: a pressure line, an interface extension tube (for example interface conduit 185), one or more adaptor(s).

Where a gases passage is described in the specification it will be appreciated that the gases passage may be a breathing gases passage for the passage of breathing gases to and/or from a patient (for example via a patient interface).

The gases passage of the medical tube assembly may be defined by a number of components of the medical tube assembly as described below.

The configurations as described relate to expiratory tubes, however it will be appreciated that the disclosure is equally applicable to inspiratory tubes, or any other tubes in a breathing circuit.

The medical tube assemblies 1 and tubes 5 are shown in at least some of the Figures as a particular length however it will be appreciated that the medical tube assemblies 1 and tubes 5 may be any length.

Throughout the specification where the term cross-sectional area of a gases passage is used, it may refer to the cross-sectional area of the gases passage in a direction perpendicular to a longitudinal axis of the gases passage. However, as described in more detail below the cross-sectional area of a corrugated portion may be defined with reference to a corrugation (for example including a crest and a trough).

The medical tube assembly 1 may be provided as part of an expiratory pathway (for example to provide a pathway for expired gases from a patient to return to a device (for example a gases source)). The expiratory pathway also forms part of the breathing circuit.

The expiratory pathway may comprise an expiratory conduit 172, a filter and a Y-piece 113.

In some configurations the expiratory pathway Y-piece 113 may be a swivel adaptor.

As described above, the disclosed medical tube assembly 1 may reduce resistance to flow (relative to tube assemblies which do not have one or more of the features and/or cross sectional areas/diameters disclosed) while balancing pneumatic compliance considerations. As described above this may be particularly important in neonatal and/or paediatric patients where small tidal volumes are delivered. In these cases, resistance to flow needs to be decreased to allow the patient to exhale through an expiratory pathway while maintaining/balancing) pneumatic compliance to ensure accurate delivery of tidal volume.

For example, the diameter/cross sectional area of the tube connection portion may be increased to a minimum diameter of about 10 mm to about 16 mm, and/or a minimum cross-sectional area of about 100 mm² to about 150 mm².

The diameter and/or cross-sectional area of the tube connection portion may be able to be increased by provision of for example an increasing gases passage cross-sectional area portion 35, 235 so as to remove a potential flow restriction and decrease the resistance to flow of the medical tube assembly 1.

In some configurations there may be a resistance to flow of the expiratory pathway (for example as defined by a pressure drop across the expiratory pathway). In some configurations the pressure drop across the expiratory pathway may be less than 1.8 cmH2O for a flow of gases at 30 Litres/minute, or 1.8 cmH2O at 15 Litres/minute, or 1.85 cmH2O at 2.5 Litres/minute. In some configurations the pressure drop across the expiratory pathway may be in compliance with ISO 5367:2014.

It will be appreciated that the medical tube assembly as described in this specification may be an expiratory tube 172 as part of a breathing circuit. The breathing circuit may comprise one or more of: a dryline 130 (which may also be called the supply tube 157) extending from, and/or configured to extend from, a gases source 105, a humidification chamber (or other humidifier flow path) configured to connect to the dry line 130 or supply tube 157, and an inspiratory tube 170 configured to connect the chamber or humidifier to a Y-piece 113, a Y-piece 113, and an expiratory conduit 172 (for example as the tube described above) configured to connect to a Y-piece 113.

In some configurations the breathing circuit may have a pneumatic compliance. In some configurations the pneumatic compliance of the breathing circuit may be less than 1.5 ml/cmH2O at 60 cmH2O. In some configurations the pneumatic compliance of the breathing circuit may be less than 4 ml/cmH2O at 60 cmH2O. In some configurations the pneumatic compliance of the breathing circuit may be less than 5 ml/cmH2O at 60 cmH2O. The pneumatic compliance may be in compliance with ISO 5367:2014.

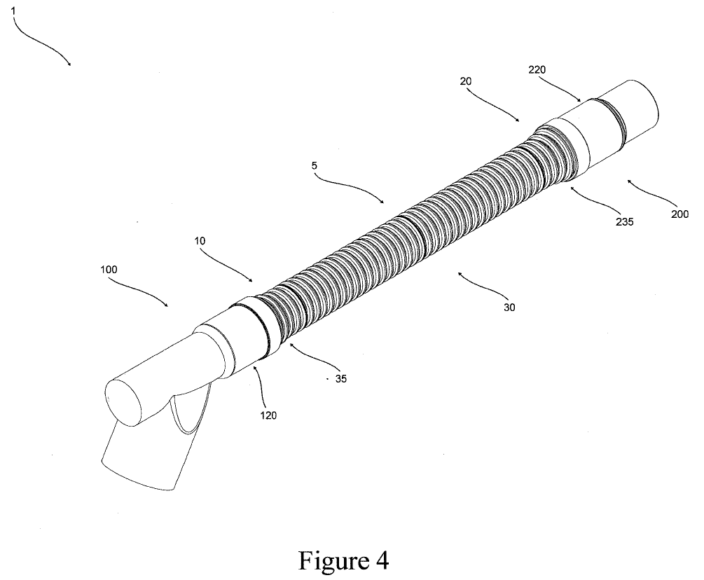

FIGS. 4-8 show an example of a medical tube assembly 1. The medical tube assembly comprises a tube 5. The tube 5 defines a gases passage 6 extending from a first end 10 of the tube 5 to a second end 20 of the tube 5.

The gases passages described may allow for the passage of gases in any direction. However, in some configurations the medical tube assembly may be provided as part of a breathing circuit and the gases may only flow in one direction in normal use.

As shown in FIGS. 4-10, the tube 5 comprises a main body portion 30, and at least one increasing gases passage cross-sectional area portion 35, 235. As described in more detail elsewhere, the at least one increasing gases passage cross-sectional area portion 35, 235 may allow for the cross-sectional area of the gases passage of the tube connection portion 110, 210 to be increased (relative to a tube without an increasing gases passage cross-sectional area portion 35, 235) to remove a potential flow restriction and decrease the resistance to flow of the medical tube assembly 1.

FIGS. 4-7 show an increasing gases passage cross-sectional area portion 35, 235 at both ends of the tube, however it will be appreciated that in some configurations, the increasing gases passage cross-sectional area portion 35, 235 may only be present at a single end of the tube. The increasing gases passage cross-sectional area portion 35, 235 increases in cross-sectional area in a direction towards the ends 10, 20 of the tube 5 it is associated with. For example, the increasing gases passage cross-sectional area portion 35 of the first end 10 of the tube 5 increases in gases passage cross-sectional area towards the first end 10 of the tube 5, and the gases passage cross-sectional area portion 235 of the second end 20 of the tube 5 increases in gases passage cross-sectional area towards the second end 20 of the tube 5.

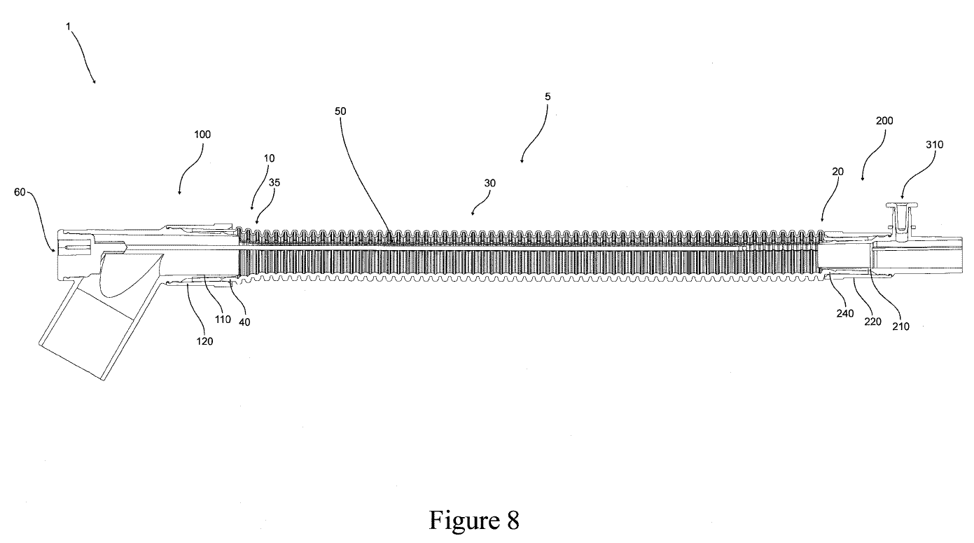

For example, in the medical tube assembly 1 as shown in FIG. 8 the increasing gases passage cross-sectional area portion 35 is only present at the first end 10 of the tube 5.

The medical tube assembly 1 as shown for example in FIGS. 4-7, comprises a first connector 100 and a second connector 200. The first connector 100 is connected to the first end 10 of the tube 5, and the second connector 200 is connected to the second end 20 of the tube 5. Each of the first connector 100 and second connector 200 define a gases passage 101, 201 extending through the connector.

The first connector 100 may be a device end connector for example configured to connect to a device (or a component which is itself connected to a device) such as a gases source (optionally the medical tube assembly is an expiratory tube). In some examples, the first connector connects to another component.

The second connector 200 may be a patient end connector for example configured to connect to a y-piece 113 and/or a patient interface 180 (or another component which is itself connected to the y-piece 113 and/or a patient interface 180). In some examples, the second connector connects to another component.

The gases passage 6 of the tube 5 in combination with the gases passage 101 of the connector 100 and the gases passage 201 of the second connector 200 defines the gases passage of the medical tube assembly.

As described in more detail below the connector 100, 200 may comprise a tube connection portion 110, 210 configured to be provided inside the tube 5 when the connector 100, 200 is connected to the tube 5.

As described in more detail below the medical tube assembly 1 may comprise a collar 120, 220. The collar 120, 220 is configured to engage an external surface of the tube 5 when the connector 100, 200 is connected to the tube 5.

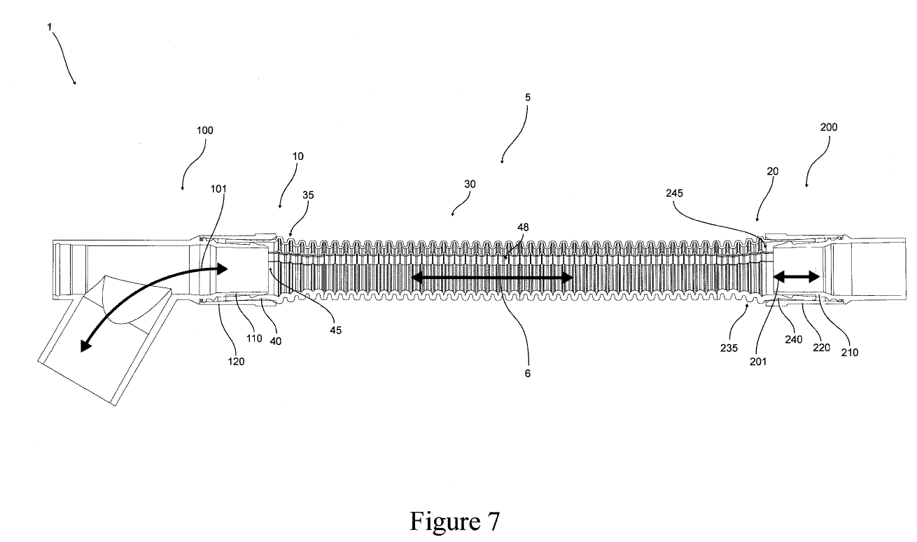

As for example shown in FIG. 7, the tube 5 is retained between the collar 120, 220 and the tube connection portion 110, 210 of the connector 100, 200. In some configurations the cuff 40, 240 of the tube 5 is retained between the collar 120, 220 and the tube connection portion 110, 210 of the connector 100, 200. This type of connection may be beneficial when the tube 5 changes dimensionally and/or in material properties when exposed to water as disclosed in more detail below.

In some configurations, the tube 5 may be compressed between the collar 120, 220 and the tube connection portion 110, 210 (when the tube is retained between the 120, 220 and the tube connection portion 110, 210).

Throughout the description the medical tube assembly 1 is described as having connectors 100, 200, and collars 120, 220 at both ends of the tube, however in some configurations, a connector 100, 200 and collar 120, 220 may be provided at a single end.

Features of the connectors 100, 200, and collars 120, 220 are described below for both connectors 100, 200, and both collars 120, 220, however it will be appreciated that each of the connectors 100, 200, and each of the collars 120, 220 may include any of the combinations of features described.

The connectors 100, 200 may be configured to connect to associated connectors so as to couple the medical tube assembly to associated connectors at each end.

The breathing gases passage of the tube may be cylindrical.

The tube as shown in the Figures is generally cylindrical-however it will be appreciated that other shaped tubes are included within the definition of the term tube, and portions of the tube may be a non-circular cross-section.

In some configurations, at least part of the tube 5 is corrugated. The corrugations may provide the tube with stiffness (which may help with pneumatic compliance of the tube) while still allowing for flexibility to allow for easy use of the tube by a user.

The corrugations may also increase a surface area of the tube 5. In configurations where the tube is a breathable material this may increase the surface area (for example the internal surface area and the external surface area) available for the passage of water molecules to the ambient environment.

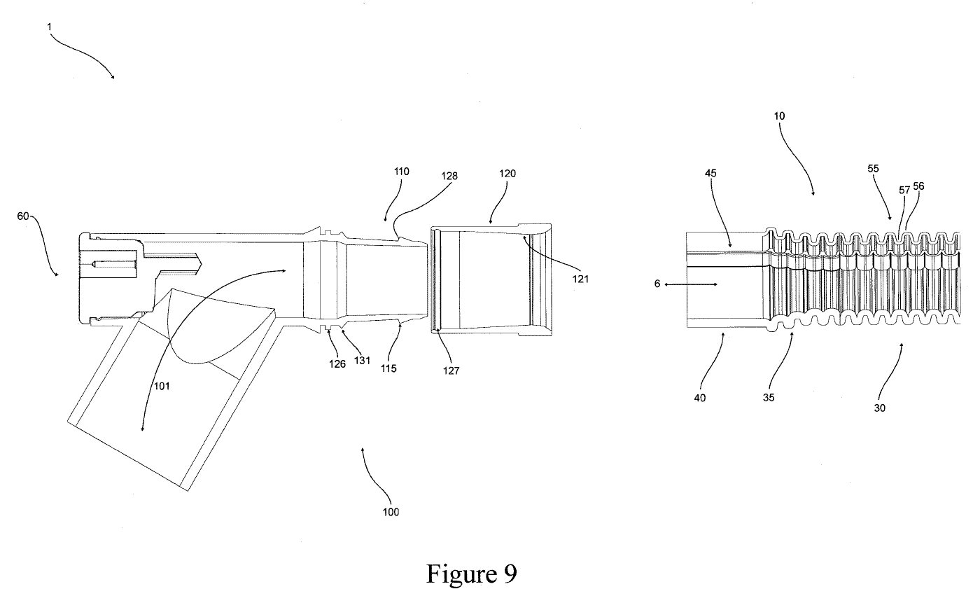

As shown in FIGS. 4-7, the main body portion 30 of tube 5 is a corrugated tube. As shown for example in FIG. 9 the corrugations are annular corrugations, however in some embodiments the corrugations may be helical.

The tube 5 comprises a plurality of crests 56 and troughs 57 (as for example shown in FIG. 9). The crests 56 and troughs 58 extend around the periphery (for example a circumference in a circular tube) of the tube and alternate along the tube (i.e., in a direction generally parallel to a longitudinal axis of the tube 5).

Referring again to FIG. 9, the corrugations may be formed by alternating crests and troughs.

It will be appreciated that in some configurations the tube is non-corrugated.

It will be appreciated that only portions of the tube may be corrugated. For example, as described below in more detail a cuff region of the tube may not be corrugated.

The tube 5 may comprise at least one rib 48. The ribs may extend longitudinally in a direction along the length of the tube (for example at least in part parallel, or parallel to a longitudinal axis of the tube 5). The ribs 48 may be the same ribs 45, 245 of the cuff regions 40, 240 for example as shown in FIGS. 9-10 and 12-13. In some configurations, the ribs 45, 245 of the cuff regions 40, 240 may be separate to the at least one rib 48.

In some configurations, the at least one rib extends longitudinally along the tube.

The ribs 48 are shown in cross section on some of the figures for example FIGS. 7-14. However, it will be appreciated that these ribs may not be included in determining any diameters and/or cross-sectional areas of the gases passage for example as shown in FIGS. 15-22.

The tube 5 may comprise one or a plurality of ribs 48. For example, the tube 5 may comprise 1 to 4 ribs, or 3 ribs (as for example shown in FIG. 16). As shown in for example FIG. 16, ribs 48 are located on an inner surface of the tube. In some configurations, the ribs 48 may be equidistantly spaced around the periphery of the gases flow passage of the tube.

As described above the cross-sectional area of the gases passage may be in a direction perpendicular to a longitudinal axis of the gases passage.

The gases passage cross-sectional area 31 of the main body portion 30 of the tube 5 may be defined by:
a) a minimum cross-sectional area of the gases passage of the main body portion of the tube (as for example an example minimum cross-sectional area of the gases passage along the length of the tube is shown in FIG. 16), or
b) a maximum cross-sectional area of the gases passage of the main body portion of the tube.

Additionally, in configurations where the main body portion of the tube is corrugated the cross-sectional area of the tube may be defined by the nominal cross-sectional area. The nominal cross-sectional area being an average of the minimum cross-sectional area of the gases passage of the main body portion of the tube and the maximum cross-sectional area of the gases passage of the main body portion of the tube.

In some configurations, the minimum cross-sectional area of the gases passage of the main body portion 30 of the tube is about 100 mm² to about 150 mm², or about 100 mm² to about 130 mm², or about 120 mm².

In some configurations, the maximum cross-sectional area of the gases passage of the main body portion 30 of the tube is about 160 mm² to about 210 mm², or about 180 mm² to about 200 mm², or about 190 mm².

In some configurations, the nominal cross-sectional area of the gases passage of the main body portion 30 of the tube is about 120 mm² to about 180 mm², or about 145 mm² to about 165 mm², or about 155 mm².

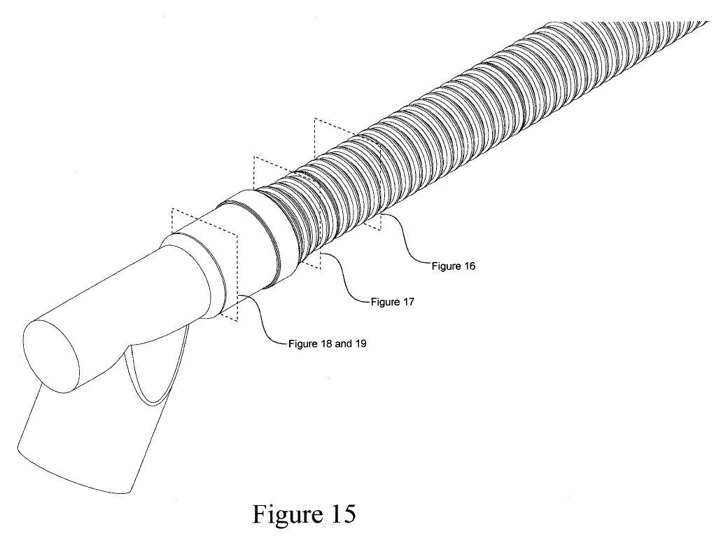

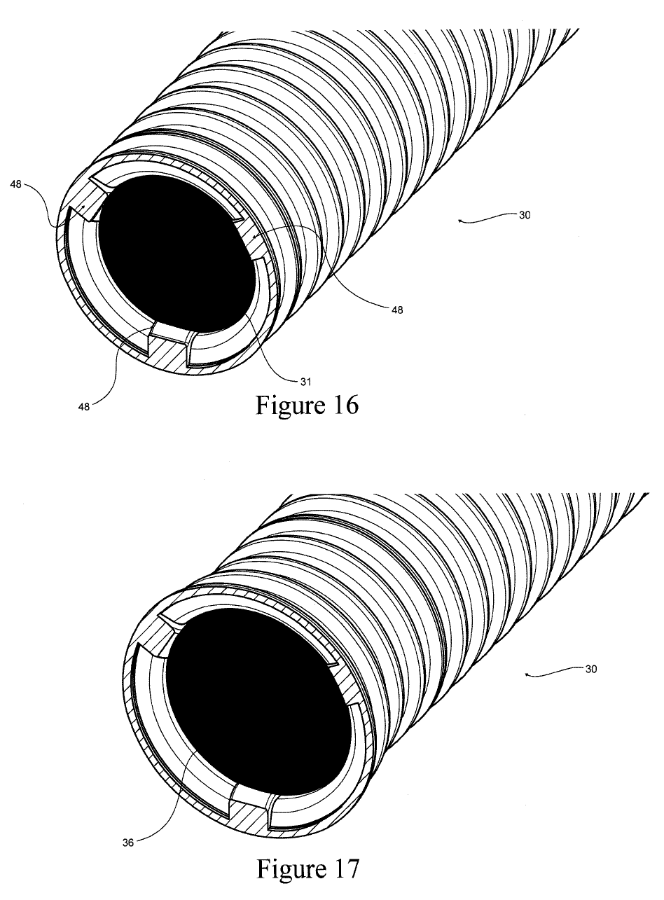

The ribs may not be considered in defining the cross-sectional area of the gases passage (for example as shown in FIGS. 16 and 17 where the ribs are not included in the cross-sectional area of the gases passage).

In some configurations, the minimum cross-sectional area of the gases passage of the main body portion 30 of the tube, not including any ribs, is about 110 mm² to about 160 mm² or about 130 mm² to about 150 mm².

In some configurations, the maximum cross-sectional area of the gases passage of the main body portion 30 of the tube, not including any ribs, is about 180 mm² to about 230 mm² or about 190 mm² to about 210 mm².

In some configurations, the nominal cross-sectional area of the gases passage of the main body portion 30 of the tube, not including any ribs, is about 140 mm² to about 200 mm².

The gases passage cross-sectional area 31 of the main body portion 30 of the tube 5 may be defined by:
a) a local minimum cross-sectional area of the gases passage of the main body portion of the tube (as for example an example minimum cross-sectional area of the gases passage along the length of the tube is shown in FIG. 16), for example when the tube is a corrugated tube the minimum cross-sectional area is defined by a single pair opposing troughs, or
b) the local maximum cross-sectional area of the gases passage of the main body portion of the tube for example when the tube is a corrugated tube the maximum cross-sectional area is defined by a single pair of opposing crests.

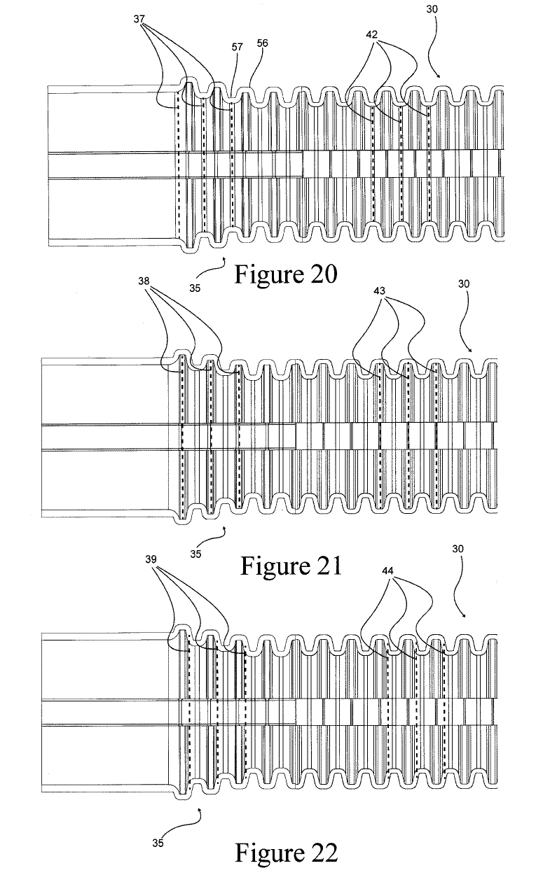

As shown by way of example in FIG. 20 the local minimum cross-sectional areas 42 of the gases passage of the main portion 30 of the tube 5 are defined by an inner surface of opposing troughs 57. In the tube of FIG. 20, each local minimum cross-sectional area 42 is the same area as a minimum cross-sectional area of the main body portion of the tube, as the local minimum cross-sectional areas 42 are all the same area along the length of the tube.

As shown by way of example in FIG. 21 the maximum local cross-sectional areas 43 of the gases passage of the main portion 30 of the tube 5 are defined by an inner surface of opposing crests 58. In the tube of FIG. 21, each local maximum cross-sectional area 42 is the same area as a maximum cross-sectional area of the main body portion of the tube, as the local maximums cross-sectional areas 42 are all the same area along the length of the tube.

Additionally, for example as shown in FIG. 22, in configurations where the main body portion of the tube is corrugated the cross-sectional area of the tube may be defined by the local nominal cross-sectional area 44 being an average of the area of the gases passage of the tube defined by a trough of the tube (for example corresponding to a local minimum) and an area of the gases passage of the tube defined by a crest of the tube (for example corresponding to a local maximum) adjacent the trough.

In some configurations, the local minimum cross-sectional area of the gases passage of the main body portion 30 of the tube is about 100 mm² to about 150 mm², or about 110 mm² to about 130 mm², or about 120 mm².

In some configurations, the local maximum cross-sectional area of the gases passage of the main body portion 30 of the tube is about 160 mm² to about 210 mm², or about 180 mm² to about 200 mm², or about 190 mm².

In some configurations, the local nominal cross-sectional area of the gases passage of the main body portion 30 of the tube is about 120 mm² to about 180 mm², or about 145 mm² to about 165 mm², or about 155 mm².

The ribs may not be considered in defining the cross-sectional area of the gases passage (for example as shown in FIGS. 16 and 17 where the ribs are not included in the cross-sectional area of the gases passage).

In some configurations, the local minimum cross-sectional area of the gases passage of the main body portion 30 of the tube, not including any ribs, is about 110 mm² to about 160 mm² or about 130 mm² to about 140 mm².

In some configurations, the local maximum cross-sectional area of the gases passage of the main body portion 30 of the tube, not including any ribs, is about 180 mm² to about 230 mm² or about 190 mm² to about 210 mm².

In some configurations, the local nominal cross-sectional area of the gases passage of the main body portion 30 of the tube, not including any ribs, is about 140 mm² to about 200 mm² or about 160 mm² to about 180 mm².

It will be appreciated that in circular cross-section tubes the diameter of the gases passage will be proportional to the cross-sectional area of the gases passage.

In FIGS. 20, 21 and 22 the cross-sectional areas 42, 43 and 44 are themselves shown in cross-section however they extend to define the cross-sectional area of the gases pathway. It will be appreciated that the cross-sectional areas as shown in these figures may also represent diameters.

The gases passage diameter 7 of the main body portion of the tube may be defined by:
a) a minimum inner diameter of the gases passage of the main body portion of the tube or
b) a maximum inner diameter of the gases passage of the main body portion of the tube.

In some configurations, the minimum inner diameter of the main body portion 30 of the tube is about 12.45 mm (±0.3 mm or ±0.5 mm or ±1.0 mm). In some configurations, the minimum inner diameter of the main body portion 30 of the tube is about 12.65 mm (±0.3 mm or ±0.5 mm or ±1.0 mm). In some configurations, the minimum inner diameter of the main body portion 30 of the tube is about 10 mm to about 16 mm, or about 12 mm to about 14 mm.

In some configurations, the maximum inner diameter of the main body portion 30 of the tube is about 15.95 mm (±0.3 mm or ±0.5 mm or ±1.0 mm). In some configurations, the maximum inner diameter of the main body portion 30 of the tube is about 16.15 mm (±0.3 mm or ±0.5 mm or ±1.0 mm). In some configurations, the maximum inner diameter of the main body portion 30 of the tube is about 14 mm to about 18 mm, or about 15 mm to about 17 mm.

Additionally, in configurations where the main body portion of the tube is corrugated the diameter of the tube may be defined by a nominal gases passage diameter, being an average of the minimum inner diameter of the gases passage of the main body portion of the tube and the maximum inner diameter of the gases passage of the main body portion of the tube.

In some configurations, the nominal diameter of the main body portion 30 of the tube is about 12 mm to about 17 mm, or about 13 mm to about 16 mm.

The gases passage diameter 7 of the main body portion 30 of the tube 5 may be defined by:
a) a local minimum diameter of the gases passage of the main body portion 30 of the tube 5, for example when the tube 5 is a corrugated tube the minimum diameter is be defined by a single pair opposing troughs, or
b) a local maximum diameter of the gases passage of the main body portion 30 of the tube 5 for example when the tube 5 is a corrugated tube the maximum diameter is be defined by a single pair opposing crests.

In configurations where the main body portion of the tube is corrugated a local nominal diameter of the gases passage may be an average of a diameter between opposing troughs of the tube and a diameter between opposing crests of the tube adjacent the trough.

In some configurations, the local minimum diameter of the main body portion 30 of the tube is about 10 mm to about 16 mm, or about 13 mm to about 15 mm, or about 12.65 mm.

In some configurations, the local maximum diameter of the main body portion 30 of the tube is about 14 mm to about 18 mm, or about 15 mm to about 17 mm, or about 16.1 mm.

In some configurations, the local nominal diameter of the main body portion 30 of the tube is about 12 mm to about 17 mm, or about 13 mm to about 16 mm, or about 15 mm.

Referring to FIG. 16, a gases passage cross-sectional area 31 of the main body portion 30 of the tube 5 is a local minimum cross-sectional area of the gases passage of the main body portion of the tube and is constant along a length of the tube. For example as shown in FIGS. 20, 21 and 22.

In some configurations, one or more of: the local minimum cross-sectional areas of the gases passage of the main body portion of the tube, the local maximum cross-sectional areas of the gases passage of the main body portion of the tube, and/or the local nominal cross-sectional areas of the gases passage of the main body portion of the tube is/are constant along the length of the main body portion.

In some cases, the local nominal diameter (or local nominal cross-sectional area) of the main body portion of the tube may be the same as the nominal diameter (or nominal cross sectional area) of the tube (for example where the corrugations are repeating and are the same size and shape.)

In some cases, the local minimum diameter (or local minimum cross-sectional area) of the main body portion of the tube may be the same as the minimum diameter (or minimum cross sectional area) of the tube (for example where the corrugations are repeating and are the same size and shape.)

In some cases, the local maximum diameter (or local maximum cross-sectional area) of the main body portion of the tube may be the same as the maximum diameter (or maximum cross sectional area) of the tube (for example where the corrugations are repeating and are the same size and shape.)

In some configurations, one or more of: the local minimum diameters of the gases passage of the main body portion of the tube, the local maximum diameters of the gases passage of the main body portion of the tube, and/or the local nominal diameters of the gases passage of the main body portion of the tube is/are constant along the length of the main body portion.

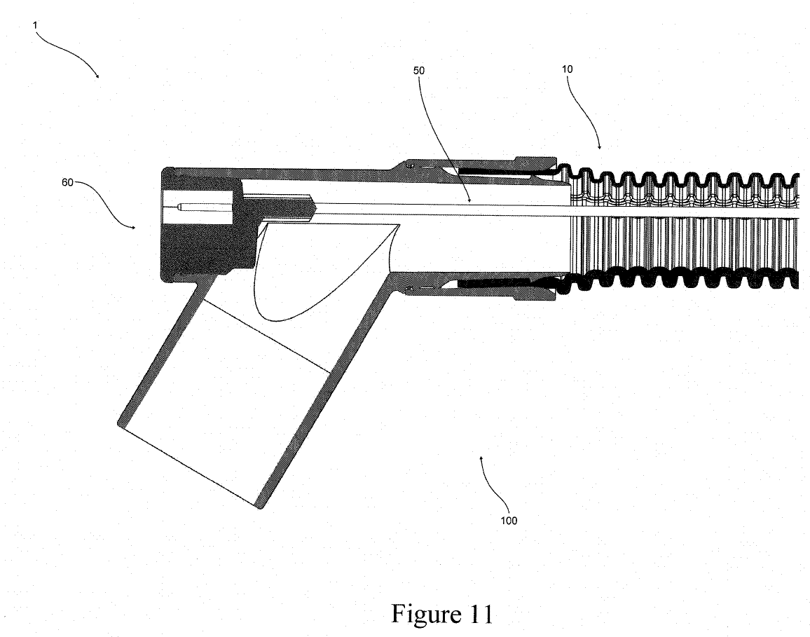

As shown in FIG. 8 and FIG. 11, the medical tube assembly 1 may comprise at least one heater wire 50 (or for example element 145 and/or 147 in FIG. 1.

The at least one heater wire 50 may be located in a wall of the tube, around the tube (for example wrapped around an external surface of the tube), or within a gases passage of the tube and/or a gases passage of the connector.

As shown in FIG. 8 the heater wire 50 extends from the electrical connector 60 at a first end 10 of the tube along a length of the tube and then loops back.

Referring to FIG. 11, the medical tube assembly 1 comprises an electrical connector 60 configured to allow for electrical connection to the at least one heater wire 50 (for example by connection with another electrical connector).

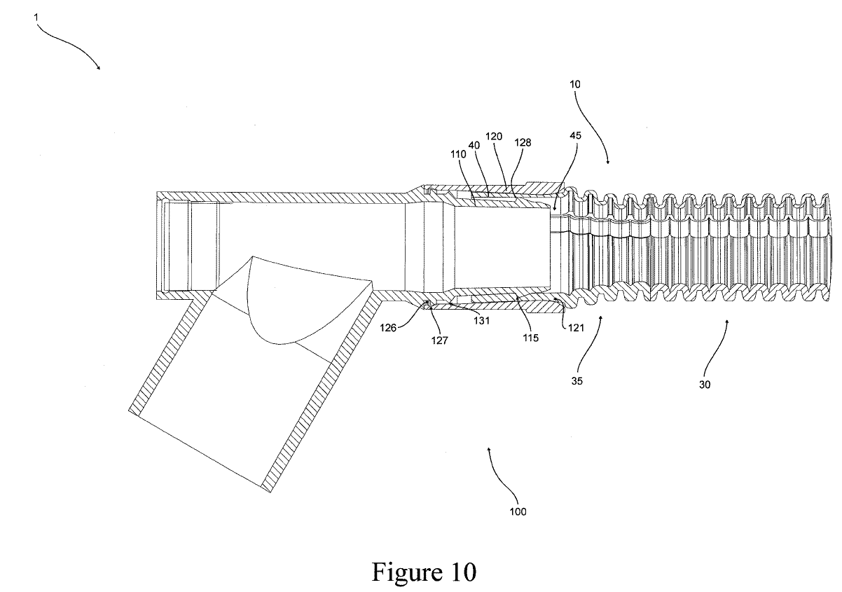

FIGS. 4, 7, 10, 14 and 15 show Figures where the electrical port 50 is not shown but may be present. It will be appreciated that if the electrical port 50 is not included the opening to allow for the port may be closed (for example as shown in FIG. 10). It will be however appreciated that the electrical port and/or heater wire can be included in any of the embodiments as shown in these Figures.

FIG. 9 also shows the electrical port but the heater wire 50 is omitted.

As shown in for example FIG. 7. The connector 100 forms an elbow. The connector 100 may form an elbow by having two portions of the connector oriented at an angle to each other. The angle may be for example about 45 degrees to about 160 degrees, or about 135 degrees, or about 120 degrees.

In some configurations, the connector 200 (for example as a second connector) is a straight connector (for example generally following an axis and/or having a co-axial inlet and outlet).

The connectors 100, 200 may connect to any component. In some configurations the medical tube assembly 1 is an expiratory tube 172.

FIG. 8 also shows a connector 200 which comprises a port 310. The port may be used to insert a sensor 135 (to measure a property of the gases flow as described above), or to sample gases from the gases passage.

The tube may be made from a combination of materials or a single material. The material, or combination of materials, may be selected in consideration of mechanical properties (such as stiffness) and/or processing properties (for extrusion).

The polymer, or blend of polymers, may comprise a thermoplastic polymer. The polymer, or blend of polymers, may comprise polyolefin and/or polyester. In some configurations, the material may comprise a copolyester thermoplastic elastomer with a polyether soft segment. The material may comprise one or more additives. In some configurations, the additives may comprise a foaming agent for example foaming agent masterbatch (comprising polyethylene and 20%, or up to about 20%, by weight of Clariant HYDROCEROL® BIH-10E). In some configurations, the additives may comprise colourants and/or polymer stabilisers.

The connectors 100, 200 may be made of a polymer, or blend of polymers.

As described above the medical tube assembly 1 may comprise an increasing gases passage cross-sectional area portion 35, 235.

The increasing gases passage cross-sectional area portion 35, 235 may allow for an increase in cross-sectional area of the gases passage at or near the first end 10 and/or the second end 20 of the tube 5 to allow for connection with the tube connection portion 110, 210 (as described in more detail below). The increasing gases passage cross-sectional area portion 35, 235 may allow for a larger gases passage of the tube connection portion 110, 210. This means that when a connection type that connects to the inside of the tube is utilised, the tube can increase in cross-sectional area so that the connector (i.e. the first connector 100 and/or second connector 200) does not introduce a flow restriction to the medical tube assembly. If the cross-sectional area of the tube did not increase to allow for an internal connector, the connector (i.e. the first connector 100 and/or second connector 200) may introduce such a flow restriction relative to the remainder of the tube as the cross-sectional area of the connector would be smaller than the cross-sectional area of the main body portion 30 of the tube 5 and therefore would introduce a flow restriction.

As described above, the increase in cross-sectional area of the tube may allow for the tube connection portion 110, 210 of the connector 100, 200 to be larger and not introduce a flow restriction (relative to the main body portion 30 of the tube 5) while still maintaining a connection type which retains the tube between a collar and tube connection portion 110, 210,

As described above, in some configurations, the tube 5 may comprise an increasing gases passage cross-sectional area portion 35 at or near a first end 10 of the tube 5. The increasing gases passage cross-sectional area portion 35 comprises an increase in gases passage cross-sectional area in a direction towards a first end 10 of the tube 5 and/or towards a cuff region 40 (described in more detail elsewhere in the specification) of the first end 10 of the tube 5.

As described above, in some configurations, the tube 5 may comprise an increasing gases passage cross-sectional area portion 235 at or near a second end 20 of the tube 5 (as a second increasing gases passage cross-sectional area portion 235). The increasing gases passage cross-sectional area portion 235 comprises an increase in gases passage cross-sectional area in a direction towards a second end 20 of the tube and/or towards a cuff region 240 (described in more detail elsewhere in the specification) of the second end 20 of the tube 5.

In some configurations, the increasing gases passage cross-sectional area portion 35, 235 is a flared portion (for example a continuous increase in cross-sectional area i.e. without interruption) or comprises a step change (for example a discontinuous increase i.e. with interruption) in gases passage cross-sectional area (for example the gases passage cross-sectional area as described in more detail below).

In some configurations, when the increasing gases passage cross-sectional area portion 35, 235 is a flared portion, the increasing gases passage cross-sectional area portion 35, 235 comprises a linear or non linear increase in cross-sectional area.

The gases passage cross-sectional area of the increasing gases passage cross-sectional area portion 35, 235 may be defined by:
a) a local minimum cross-sectional area of the gases passage the increasing gases passage cross-sectional area portion 35, 235 (as for example shown as local minimum cross-sectional area 36 in FIG. 17) for example when the tube 5 is a corrugated tube the minimum diameter is defined by a single pair opposing troughs, or
b) a local maximum cross-sectional area of the gases passage of the increasing gases passage cross-sectional area portion 35, 235.

In some configurations, the increasing gases passage cross-sectional area portion 35, 235 may increase from a local minimum cross-sectional area of about 100 mm² to about 150 mm² to a local minimum cross-sectional area of about 140 mm² to about 180 mm². In some configurations, the increasing gases passage cross-sectional area portion 35, 235 may increase from a local minimum cross-sectional area of about 110 mm² to about 140 mm² to a local minimum cross-sectional area of about 150 mm² to about 170 mm².

In some configurations, the increasing gases passage cross-sectional area portion 35, 235 may increase from a local minimum cross-sectional area of 120 mm² to a local minimum cross-sectional area of about 170 mm².

In some configurations, the increasing gases passage cross-sectional area portion 35, 235 may increase from a local maximum cross-sectional area of about 160 mm² to about 210 mm² to a local maximum cross-sectional area of about 220 mm² to about 280 mm².

In some configurations, the increasing gases passage cross-sectional area portion 35, 235 may increase from a local maximum cross-sectional area of about 170 mm² to about 200 mm² to a local maximum cross-sectional area of about 240 mm² to about 260 mm².

In some configurations, the increasing gases passage cross-sectional area portion 35, 235 may increase from a local maximum cross-sectional area of 190 mm² to a local maximum cross-sectional area of 250 mm².

The ribs may not be considered in defining the cross-sectional area of the gases passage (for example as shown in FIGS. 16 and 17 where the ribs are not included in the cross-sectional area of the gases passage).

In some configurations, the increasing gases passage cross-sectional area portion 35, 235 may increase from a local minimum cross-sectional area, not including any ribs, of about 110 mm² to about 160 mm² to a local minimum cross-sectional area, not including any ribs, of about 150 mm² to about 200 mm².

In some configurations, the increasing gases passage cross-sectional area portion 35, 235 may increase from a local minimum cross-sectional area, not including any ribs, of about 120 mm² to about 150 mm² to a local minimum cross-sectional area, not including any ribs, of about 160 mm² to about 190 mm².

In some configurations, the increasing gases passage cross-sectional area portion 35, 235 may increase from a local minimum cross-sectional area, not including any ribs, of 125 mm² to a local minimum cross-sectional area, not including any ribs, of 175 mm².

In some configurations, the increasing gases passage cross-sectional area portion 35, 235 may increase from a local maximum cross-sectional area, not including any ribs, of about 180 mm² to about 230 mm² to a local maximum cross-sectional area, not including any ribs, of about 240 mm² to about 300 mm².

In some configurations, the increasing gases passage cross-sectional area portion 35, 235 may increase from a local maximum cross-sectional area, not including any ribs, of about 190 mm² to about 220 mm² to a local maximum cross-sectional area, not including any ribs, of about 250 mm² to about 290 mm².

In some configurations, the increasing gases passage cross-sectional area portion 35, 235 may increase from a local maximum cross-sectional area, not including any ribs, of 200 mm² to a local minimum cross-sectional area, not including any ribs, of 270 mm².

As shown by way of example in FIG. 20 various local minimum cross-sectional areas 37 of the gases passage of the increasing gases passage cross-sectional area portion 35, 235 are defined by inner surfaces of a single pair of opposing troughs 57. FIG. 20 illustrates the increasing cross-sectional area of the gases passage (defined by the local minimum cross-sectional area 37 of the gases passage) of the increasing gases passage cross-sectional area portion 35, 235, towards the end 10, 20 of the tube 5.

As shown by way of example in FIG. 21 various maximum local cross-sectional areas 38 of the gases passage of the increasing gases passage cross-sectional area portion 35, 235 are defined by inner surfaces of a single pair of opposing crests 58. FIG. 21 illustrates the increasing cross-sectional area of the gases passage (defined by the maximum local cross-sectional areas 38 of the gases passage) of the increasing gases passage cross-sectional area portion 35, 235, towards the end 10, 20 of the tube 5.

Additionally, for example as shown in FIG. 22, in configurations where the increasing gases passage cross-sectional area portion 35, 235 of the tube 5 is corrugated the cross-sectional area of the increasing gases passage cross-sectional area portion 35, 235 may be defined by a local nominal cross-sectional area being an average of the area of the gases passage of the increasing gases passage cross-sectional area portion 35, 235 defined by a trough of the increasing gases passage cross-sectional area portion 35, 235 and an area of the gases passage of the tube defined by a crest of the increasing gases passage cross-sectional area portion 35, 235 adjacent the trough. FIG. 22 illustrates various nominal local cross-sectional areas 39 of the gases passage of the increasing gases passage cross-sectional area portion 35, 235. FIG. 22 illustrates the increasing cross-sectional area of the gases passage (defined by the nominal local cross-sectional areas 38 of the gases passage) of the increasing gases passage cross-sectional area portion 35, 235, towards the end 10, 20 of the tube 5.

In some configurations, the increasing gases passage cross-sectional area portion 35, 235 may increase from a local nominal cross-sectional area of about 120 mm² to about 180 mm² to a local nominal cross-sectional area of about 180 mm² to about 250 mm².

In some configurations, the increasing gases passage cross-sectional area portion 35, 235 may increase from a local nominal cross-sectional area of about 130 mm² to about 170 mm² to a local nominal cross-sectional area of about 190 mm² to about 240 mm².

In some configurations, the increasing gases passage cross-sectional area portion 35, 235 may increase from a local nominal cross-sectional area, not including any ribs, of 160 mm² to a local nominal cross-sectional area, not including any ribs, of 220 mm².

When the increasing gases passage cross-sectional area portion 35, 235 is corrugated the increase in gases passage cross-sectional area may be linear or nonlinear across a plurality of corrugations.

In FIGS. 20, 21 and 22 the cross-sectional areas 37, 38 and 39 are themselves shown in cross-section however they extend to define the cross-sectional area of the gases pathway. It will be appreciated that the cross-sectional areas as shows in these figures may also represent diameters.

The gases passage diameter of the increasing gases passage cross-sectional area portion 35, 235 of the tube may be defined by:
a) a local minimum inner diameter of the gases passage of the increasing gases passage cross-sectional area portion, or
b) a local maximum inner diameter of the gases passage of the increasing gases passage cross-sectional area portion.

In some configurations, the minimum local inner diameter of the increasing gases passage cross-sectional area portion 35, 235 of the tube increases from about 12.45 mm (±0.3 mm or ±0.5 mm or ±1.0 mm) to about 14.75 mm (±0.3 mm or ±0.5 mm or ±1.0 mm).

In some configurations, the maximum local inner diameter of the increasing gases passage cross-sectional area portion 35, 235 of the tube increases from about 15.95 mm (±0.3 mm or ±0.5 mm or ±1.0 mm) to about 18.25 mm (±0.3 mm or ±0.5 mm or ±1.0 mm).

In some configurations, the increasing gases passage cross-sectional area portion 35, 235 may increase from a minimum local inner diameter of about 10 mm to a minimum local inner diameter of about 17 mm.

In some configurations, the increasing gases passage cross-sectional area portion 35, 235 may increase from a minimum local inner diameter of about 12 mm to a minimum local inner diameter of about 15 mm.

In some configurations, the increasing gases passage cross-sectional area portion 35, 235 may increase from a minimum local inner diameter of about 12.65 mm to a minimum local inner diameter of about 14.95 mm.

In some configurations, the increasing gases passage cross-sectional area portion 35, 235 may increase from a minimum local inner diameter of about 10 mm to about 16 mm to a minimum local inner diameter of about 12 mm to about 17 mm.

In some configurations, the increasing gases passage cross-sectional area portion 35, 235 may increase from a maximum local inner diameter of about 14 mm to a maximum local inner diameter of about 20 mm.

In some configurations, the increasing gases passage cross-sectional area portion 35, 235 may increase from a maximum local inner diameter of about 16 mm to a maximum local inner diameter of about 19 mm.

In some configurations, the increasing gases passage cross-sectional area portion 35, 235 may increase from a maximum local inner diameter of about 16.10 mm to a maximum local inner diameter of about 18.45 mm.

In some configurations, the increasing gases passage cross-sectional area portion 35, 235 may increase from a maximum local inner diameter of about 14 mm to about 18 mm to a maximum local inner diameter of about 16 mm to about 20 mm.

Additionally, in configurations where increasing gases passage cross-sectional area portion 35, 235 of the tube is corrugated the cross-sectional area of the increasing gases passage cross-sectional area portion 35, 235 may be defined by a local nominal gases passage diameter being an average of the diameter between opposing troughs of the tube and a diameter between opposing crests of the tube adjacent the opposing troughs.

In some configurations, the increasing gases passage cross-sectional area portion 35, 235 may increase from a local nominal gases passage diameter of about 12 mm to about 17 mm to a local nominal gases passage diameter of about 14 mm to about 19 mm.

In some configurations, the increasing gases passage cross-sectional area portion 35, 235 may increase from a local nominal gases passage diameter of about 12 mm to about 12 mm to a local nominal gases passage diameter of about 15 mm to about 18 mm.

In some configurations, the increasing gases passage cross-sectional area portion 35, 235 may increase from a local nominal gases passage diameter of about 12 mm to about 17 mm to a local nominal gases passage diameter of about 14 mm to about 18.5 mm.

In some configurations, the increasing gases passage cross-sectional area portion 35, 235 may increase from a local nominal gases passage diameter of about 14.4 mm to a local nominal gases passage diameter of 16.7 mm.

In some configurations, the increasing gases passage cross-sectional area portion 35, 235 may comprise a step change increase. In some configurations, where the increasing gases passage cross-sectional area portion 35, 235 is corrugated the step increase could occur in a single corrugation.

For example, when the gases passage is circular as shown in FIGS. 4-10, the gases passage 6 of the tube 5 in the increasing gases passage cross-sectional area portion 35 is defined by at least a diameter of the gases passage 6 of the tube 5 in the increasing gases passage cross-sectional area 35, 235. The increasing gases passage cross-sectional area portion 35, 235 may therefore have an increasing diameter.

The increasing gases passage cross-sectional portion 35 at or near the first end 10 of the tube 5 may comprise an increase in gases passage diameter in a direction towards a first end 10 of the tube 5 and/or towards a or the cuff region 40 of the first end 10 of the tube 5.

The increasing gases passage cross-sectional portion 235 at or near the second end 20 of the tube may comprise an increase in gases passage diameter in a direction towards a second end 20 of the tube 5 and/or towards a or the cuff region 240 of the second end 20 of the tube 5.

As described above, the increasing gases passage cross-sectional area portion 35, 235 may comprise a linear or non linear increase in diameter of the gases passage along a length of the increasing gases passage cross-sectional area portion 35, 235.

As described above, the increasing gases passage cross-sectional area portion 35, 235 may comprise a step change increase of the gases passage.

The increasing gases passage cross-sectional area portion 35 at or near the first end 10 of the tube 5 extends from a first location located away from (for at a distance from) the first end 10 of the tube 5 to a location near, or at the first end 10 of the tube 5. The increasing gases passage cross-sectional area portion 35 at or near the first end 10 of the tube 5 extends from a first location located away from the first end 10 of the tube 5 to a location near, or at the first end 10 of the tube 5.

As shown in for example FIGS. 4-10, the increasing gases passage cross-sectional area portion 35, 235 extends from the main body portion 30 of the tube 5.

The increasing gases passage cross-sectional area portion 35 extends from the main body portion 30 towards a first end 10 of the tube 5, and the increasing gases passage cross-sectional area portion 235 extends from the main body portion 30 towards a second end 20 of the tube 5. However, as described elsewhere in the specification it will be appreciated that in some examples only one end of the tube may have an associated increasing gases passage cross-sectional area portion 35, 235 (for example as shown in FIG. 8).

In some configurations, for example as shown in FIGS. 4-10 the increasing gases passage cross-sectional area portion 35, 235 extends from the main body portion towards the cuff region 40, 240.

The increasing gases passage cross-sectional area portion 35 at or near the first end 10 of the tube 5 comprises an increasing cross-sectional area of the gases passage of the tube 5 in a direction from the main body 30 of the tube 5 towards the first end 10 of the tube 5.

The increasing gases passage cross-sectional area portion 235 at or near the second end 20 of the tube 5 comprises an increasing cross-sectional area of the gases passage of the tube 5 in a direction from the main body 30 of the tube 5 towards the second end 20 of the tube 5.

As shown in for example FIGS. 4-10, the increasing gases passage cross-sectional area portion 35, 235 of the tube 5 is/are corrugated. However, in other configurations one or both of the increasing gases passage cross-sectional area portion 35, 235 of the tube 5 may not be corrugated.

In configurations where increasing gases passage cross-sectional area portion 35, 235 is corrugated. The increasing gases passage cross-sectional area portion 35, 235 may be defined by a number of corrugations. The number of corrugations may be about 1 to about 10 corrugations, or about 2 to about 7 corrugations, or about 2 to about 5 corrugations, or about 2 to about 2.5 corrugations. For example as shown in FIGS. 4-10, the increasing gases passage cross-sectional area portion 35 is about 2 to about 2.5 corrugations.

As shown in FIG. 7 the main body portion of the tube transitions into the increasing gases passage cross-sectional area portion 35 near the end of the first end of the tube.

As for example shown in FIG. 7, when the tube 5 is connected to the connector 100, 200 an end of the increasing gases passage cross-sectional area portion 35, 235 is located at an end of the tube connection portion 110, 120, the connector 100, 200 and/or the collar 120, 220 when the tube 5 is connected to the connector 100, 200.

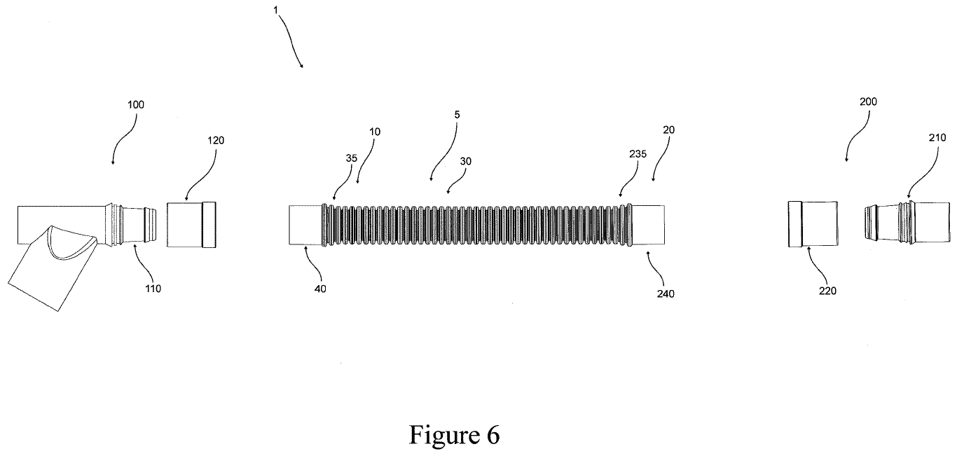

As for example as shown in FIGS. 6-7, the tube comprises a cuff region 40, 240. The cuff region 40 is located at or near the first end 10 of the tube 5. The cuff region 240 is located at or near the second end 20 of the tube 5. As shown in the figures both ends of the tube 5 have associated cuff regions 40, 240, however, in some configurations only a single end of the tube 5 will comprise a cuff region.

The cuff region 40, 240 extends from the increasing gases passage cross-sectional area portion 35, 235.

In the embodiment of FIG. 8 the cuff region 240 of the second connector 200 extends from the main body portion 30 of the tube 5.

The cuff region 40 extends towards a first end 10 of the tube 5.

The cuff region 40 of the first end 10 of the tube 5 is configured to engage with the tube connection portion 110 of the connector 100.

The cuff region 240 extends towards a second end 10 of the tube 5.

The cuff region 240 of the second end 20 of the tube 5 is configured to engage with the tube connection portion 210 of the second connector 200.

The cuff region 40, 240 comprises at least one rib 45, 245 for example as shown in FIGS. 9-10 and 12-13.

The cuff region 40, 240 comprises at least one rib 45, 245 orientated at least in part parallel to, or parallel to, a longitudinal axis of the tube 5.

The rib 45, 245 of the cuff region 40, 240 may be the same rib 48 as described above and shown in the Figures, or be a separate rib.

As shown for example in FIGS. 10, the at least one rib 45, 245 is configured to engage with a retention feature 121, 221 of the collar 120, 220 and/or the tube connection portion 110, 210 of the connector 100, 200.

The at least one rib 45, 245 may provide for a portion which the collar 120, 220 and/or the tube connection portion 110, 210 can engage with to aid in retaining the tube 5. For example, the rib 45, 245 may be compressed between the collar 120, 220 and/or the tube connection portion 110, 210.

In some configurations, for example as shown in FIG. 7, the rib 45 extends from the cuff region 40 of the first end of the tube, along the length of the tube 5 to the cuff region 240 of the second end of the tube 20 as a single rib 45, 245. In some configurations, the rib may be discontinuous.

The cuff region 40, 240 may have a larger maximum and/or minimum and/or nominal inner cross-sectional area than a or the gases passage cross-sectional area of the main body portion 30 of the tube 5 (for example any of the maximum cross-sectional area, minimum cross sectional area, nominal cross sectional area, local maximum cross-sectional area, local minimum cross sectional area and/or nominal local cross sectional area of the gases passage of the main body portion of the tube).

The cuff region 40, 240 may have a larger inner diameter than a or the gases passage cross-sectional area of the main body portion 30 of the tube 5.

In some configurations the minimum inner diameter of the cuff is about 16.10 mm (±0.3 mm or ±0.5 mm or ±1.0 mm).

In some configurations the minimum inner diameter of the cuff is about 18.45 mm (±0.3 mm or ±0.5 mm or ±1.0 mm).

In some configurations the minimum inner diameter of the cuff is about 14 mm to about 19 mm.

In some configurations the minimum inner cross-sectional area, not including the ribs, of about 170 mm² to about 220 mm², or about 190 mm² to about 210 mm², or about 200 mm².

In some configurations the minimum inner cross-sectional area 78 of about 180 mm² to about 230 mm²., or about 190 mm² to about 210 mm², or about 200 mm².

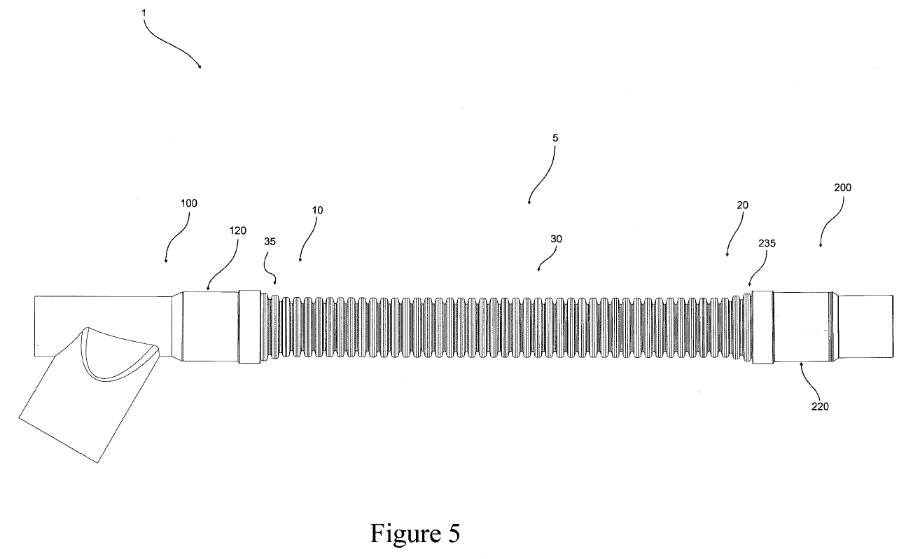

As shown in FIGS. 4-5, the cuff region 40, 240 is non-corrugated. In some configurations, the cuff region 40, 240 may be corrugated.

In some examples, the cuff region 40, 240 may have a larger maximum and/or minimum and/or nominal inner cross-sectional area than the gases passage cross-sectional area (and optionally any one of, or all of the local minimum cross-sectional areas) of an adjacent increasing gases passage cross-sectional area portion 35 (for example any of the maximum cross-sectional area, minimum cross sectional area, nominal cross sectional area, local maximum cross-sectional area, local minimum cross sectional area and/or nominal local cross sectional area of the gases passage of the main body portion of the tube.)

As described above the medical tube assembly 1 may comprise a collar 120, 220. The collar 120, 220 is configured to engage an external surface of the tube 5 when the connector 100, 200 is connected to the tube 5.

The connection between the connector 100, 200 and the tube 5 where the tube 5 is retained between the collar 120, 220 and the tube connection portion 110, 210 may aid in retaining tubes which change dimensionally and/or in material property when exposed to water (which may be as a consequence of the provision of therapy). For example, when exposed to liquid water and/or humidity a material may increase dimensionally (for example by swelling or otherwise expanding) and/or may get softer and more easily deformable (for example due to applied forces). This may present problems with pneumatic leaks and/or disconnections of the tube and connector. As discussed above a water absorbing material may be one of these types of material as described in more detail elsewhere in the specification.

A connection type where the tube 5 is retained between the collar 120, 220 and the tube connection portion 110, 210 (and optionally with one or more additional attachment and/or sealing features as described below) may allow for changes to the material without disconnection as the tube is retained by compression of the tube 5 by the collar 120, 220 and the tube connection portion 110, 210.

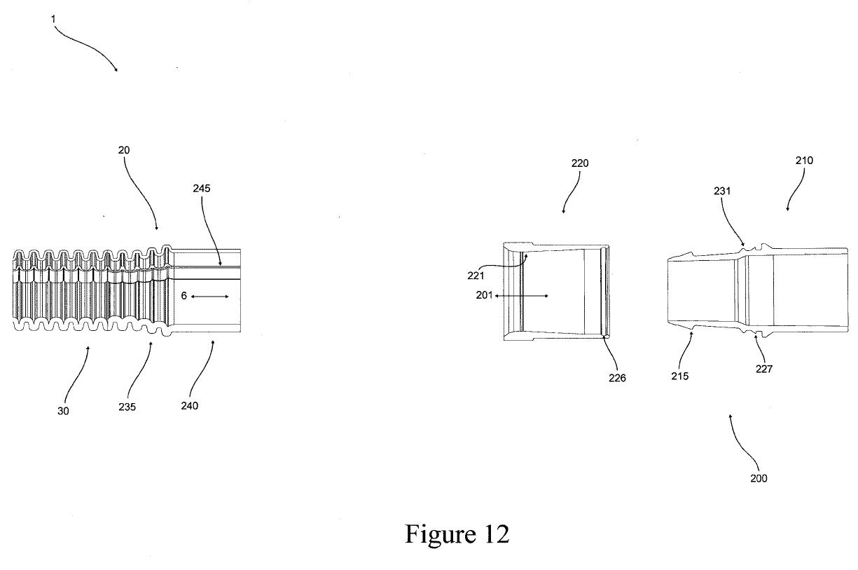

As for example shown in FIGS. 9 and 12, an internal surface of the collar 120, 220 comprises a retention feature, the retention 115, 215 feature configured to engage with an external surface of the tube 5 to aid in retaining the tube 5 on the connector 100, 200.

The connector 100, 200 and/or the collar 120, 220 comprise at least one attachment feature, the attachment feature configured to retain the collar 120, 220 to the connector 100, 200.

In some configurations, the at least one attachment feature may comprise at least one attachment protrusion 126, 226.

In some configurations, the at least one attachment feature comprises an attachment protrusion 126, 226 configured to engage a complementary recess 127, 227.

In some configurations, the attachment protrusion 126, 226 is located on one of the connector 100, 200 or the collar 120, 220, and the recess is located on the other of the connector or the collar. For example as shown in FIGS. 9 and 12 the attachment protrusion 126, 226 is located on the connector and the recess 127, 227 is located on the collar 120, 220.

In some configurations, the tube connection portion 110, 210 of the connector 100, 200 may comprise the attachment feature (for example the attachment protrusion 126, 226 or the recess 127, 227).

As shown in FIGS. 7, 10 and 13, the collar 120, 220 is configured to engage an external surface of a or the cuff region 40, 240 when the connector 100, 200 is connected to the tube 5.

The cuff region 40, 240 of the tube 5 is retained between the collar 120, 220 and the tube connection portion 110, 210.

In some configurations the tube connection portion 110, 210 of the connector 100, 200 may comprise a retention feature 115, 215. As shown in FIGS. 9-10 and 12-13, the retention feature 115, 215 may be configured to aid in retaining the tube 5 on the connector 100, 200.

In some configurations the retention feature 115, 215 may be configured to engage with an internal surface of the tube 5. In some configurations the retention feature 115, 215 may be configured to engage with an internal surface of the cuff region 40.

The retention features 115, 215 may help to retain the tube 5 on tube connection portion 110, 210.

In some configurations the retention feature 115, 215 may comprise at least one retention protrusion. The at least one retention protrusion may be configured to extend at least in part circumferentially around the tube connection portion 110, 210. As shown in FIGS. 9-10 and 12-13, the at least one retention protrusion is configured to extend around an external surface of the tube connection portion. In some configurations the retention feature 115, 215 may comprise a helical retention protrusion.

In some configurations the tube connection portion 110 and the retention feature 115 of the connector 100 may form a barb.

The retention feature 115, 215 comprises a ramp. The ramp increases in height in a direction away from an end of the tube connection portion 110, 120 configured to engage the tube. The ramp may provide an easy way to push the tube 5 onto the tube connection portion 110, 210 during assembly. The ramp may comprise a descending portion 128 (for example as shown on FIGS. 9 and 10) which provides a resistance to removing the tube 5 when the tube 5 is connected. The descending portion 128 may aid in retaining the tube 5 on the connector 100, 200.

In some configurations, the connection between the tube and the tube connection portion 110, 210 may be a permanent connection. A permanent connection may be for example a connection which cannot be disconnected without damaging the tube assembly.

In some configurations the tube connection portion 110, 210 and/or the collar 120, 220 may comprise a sealing feature. The sealing feature may be configured to provide a pneumatic seal between the collar 120 and the tube connection portion 110.

In some configurations the sealing feature may comprise at least one sealing protrusion 131, 231. For example, as shown in FIGS. 9-10 and 12-13, the at least one sealing protrusion 131, 231 may be provided on the tube connection portion 110, 210. In some configurations, the at least one sealing protrusion 131, 231 may be configured to extend at least in part circumferentially around the tube connection portion 110, 210. In some configurations, the at least one sealing protrusion is configured to extend around an external surface of the tube connection portion. By way of further example, the at least one sealing protrusion 131, 231 may additionally, or alternatively be provided on the collar 120, 220 and extend toward the tube connection portion 110, 210. In some configurations, the at least one sealing protrusion 131, 231 may be configured to extend at least in part circumferentially around the collar 120, 220.

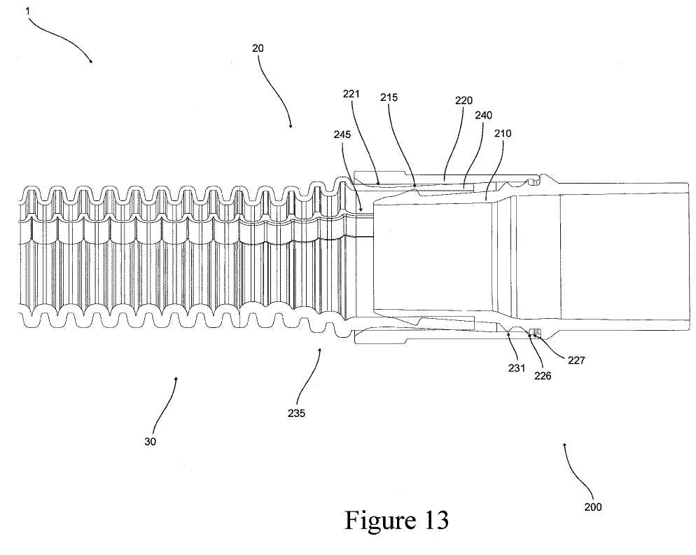

In some configurations, for example as shown in FIG. 13, the sealing feature may be located between the retention feature 115, 215 and the at least one attachment feature (for example attachment protrusion 126, 226 or the recess 127, 227).

The sealing feature may be located closer to the attachment feature than the retention feature.

As described above the tube 5 may comprise a water absorbing material. When humidified gases are provided through the tube 5 the water absorbing material of the tube 5 may absorb condensate and/or water vapour from the gases. The water absorbing material may increase in size as it absorbs water which may cause the tube 5 and/or portion of the tube 5 to increase in size. The tube 5 increasing in size when it absorbs water may cause the interface between the tube 5 and connector 100, 200 to leak. This may be caused by the change in diameter of the tube 5 introducing a gases leak path between the connector 100, 200 and the tube 5. A connection type which comprises a tube connection portion 110, 210 configured to be provided inside the tube 5 when the connector 100, 200 is connected to the tube 5, and a collar 120, 220 configured to engage an external surface of the tube 5 when the connector 100, 200 is connected to the tube 5 may reduce the chance of gases leaking as the tube 5 is retained between the tube connection portion 110, 210 and the collar 120, 220, and expansion of the tube 5 does not cause the generation of a leak path.

The water absorbing material may also get softer as it absorbs water, which may make it more difficult for the connector to retain the tube 5 as it will deform more easily. Having the tube retained between the collar 120, 220 and tube connection portion 110, 210 may help to reduce the risk of the tube 5 becoming disconnected when the tube softens.

The tube (and for example the tube wall) may be formed at least in part by the water absorbing material.

In some configurations, the wall of the tube is formed, at least in part, by the breathable material.

In some configurations, the wall of the tube is formed entirely by the breathable material.

The water absorbing material may be a breathable material.

“Breathable material” refers to a non-porous permeable material that allows the passage of water molecules through a monolithic wall of the permeable material via solution-diffusion mechanism, without allowing the bulk passage of liquid water or bulk flow of respiratory gases all the way through the wall. It should be appreciated by one of skill in the art that the water molecules in the wall are molecularly dispersed in the media, and are therefore without a state (solid, liquid, or gas), although they are sometimes referred to in the art as vapor (e.g., the rate of transfer is often referred to as a moisture vapor transmission rate (MVTR) or the like). It should further be appreciated that a monolithic wall does not contain open channels or pores from one major surface to another, such that pathogens could be carried through such channels alongside air or liquid water drops via the pore flow mechanism. However, this definition is not intended to exclude a tube formed from such a breathable material which may have one or more holes provided through the material, such as might arise from a manufacturing defect for example, which may result in negligible pore flow which does not materially affect the overall performance of the tube and compliance with ISO 5367:2014. It should yet further be appreciated that, like all polymers, some small molecule transport of respiratory gases (such as oxygen, carbon dioxide, nitrogen or helium) may occur in trace or de minimis amounts (i.e., not “bulk” flow), which, for a breathable material as defined herein, would typically be at a rate at least an order of magnitude lower than that for water molecules. Furthermore, of particular relevance for respiratory gases being delivered to or from a patient, such small molecule transport of respiratory gases would be of an amount less than that allowed for compliance with the relevant standards, for example, in the leakage test of ISO 5367:2014, which is hereby incorporated by reference in its entirety, at Section 5.4 tested via the method set out in Annex E.

FIGS. 4-7 show an example of a medical tube assembly 1 having a first connector 100 and second connector 200).

The first connector 100 being configured to connect to the first end 10 of the tube 5.

The first connector 100 comprises a gases passage defined by a tube connection portion 110. The tube connection portion 110 is configured to engage with the first end of the tube.

The first connector 100 also comprises a second portion gases passage defined by a second portion of the first connector 100.

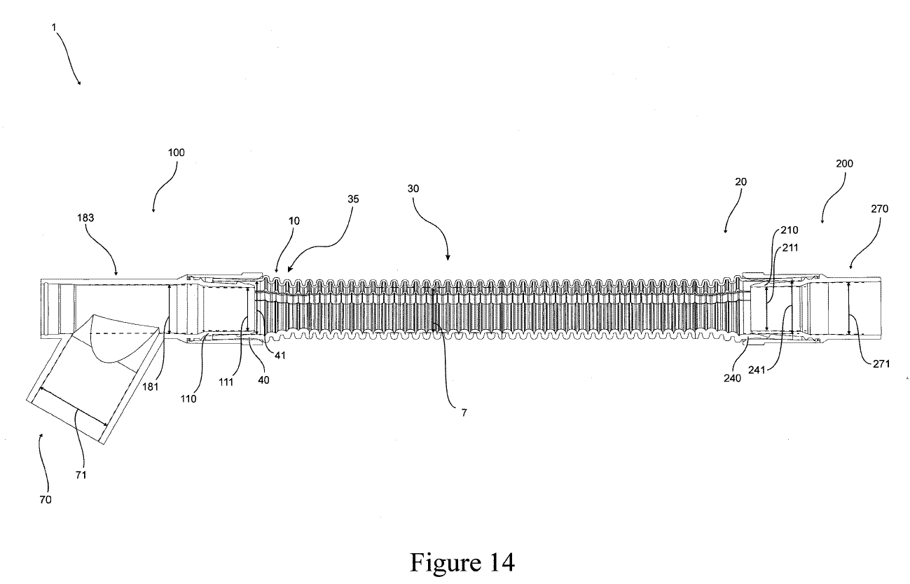

In some configurations, for example as shown in FIG. 14, the second portion of the first connector 100 may be an associated connector portion 70 configured to engage with a first associated connector. Alternatively, as shown in FIG. 14, the second portion may be an intermediate portion 183 located between the tube connection portion 110 of the first connector and an associated connector portion 70 configured to engage with a first associated connector (for example a device connector).

As shown in FIG. 14, the first connector 100 comprises an intermediate portion 183 located between the tube connection portion 110 of the first connector 100 and an associated connector portion 70 configured to engage with a first associated connector.

The second connector 200 comprises a gases passage defined by the tube connection portion 210. The tube connection portion 210 is configured to engage with the second end of the tube.

The second connector 200 also comprises a second portion gases passage defined by a second portion of the second connector 200.

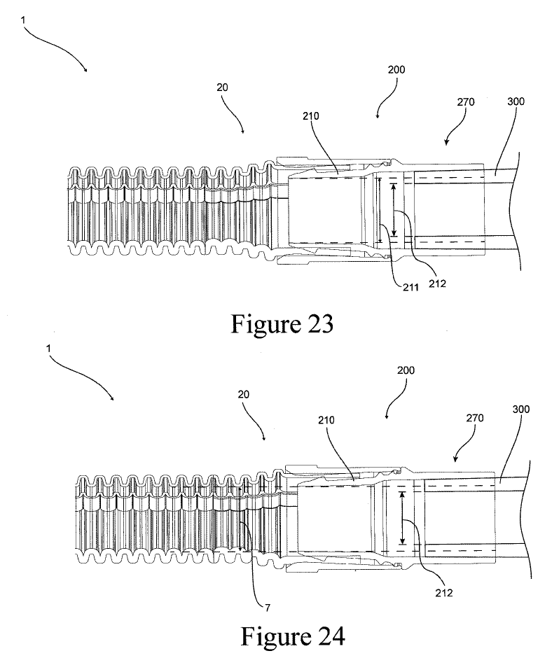

In some configurations, the second portion of the second connector 200 may be an associated connector connection portion 270 configured to engage with a second associated connector 300 (for example as shown in FIGS. 23-24). Alternatively, the second portion may be an intermediate portion 183 located between the tube connection portion 110 of the second connector and an associated connector portion 270 configured to engage with a second associated connector.

As described above, the tube 5 has a gases passage which has a cross-sectional area. Also as described above the tube may have a main body portion, the main body portion having a gases passage. In some configurations, the main body portion of the tube defines the entire tube (for example where the tube does not include a cuff region 40, 240 and/or any increasing diameter portions).

The medical tube assembly 1 may define a gases passage. The gases passage of the medical tube assembly may be defined by at least: the first connector 100, tube 5 and second connector 200.

FIG. 14 shows the diameters of various parts of the gases flow passage. FIGS. 15 19 show the cross-sectional areas of various parts of the gases flow passage.

As described above, the cross-sectional area of the gases passage may be defined at least in part by the diameter of the gases passage.

FIG. 14 shows by way of example the cross-sectional diameters of the gases passages as minimum diameter defined by minimum diameter of the gases passage. As the embodiment as shown in FIG. 14 has a circular gases passage the minimum cross-sectional area of the gases passage is based on the minimum diameter of the gases passage, and when describing the example as shown in FIG. 14 the term cross-sectional area is used. However, given the relationship between the cross-sectional area and diameter for this example, the term cross-sectional area could be used interchangeably with the term diameter.

It will be appreciated that in other examples, the diameter and/or cross-sectional area of the gases passage may be defined by for example the maximum diameters and/or cross-sectional areas of the gases passage along the length of the tube or nominal diameter and/or cross-sectional areas of the gases passage along the length of the tube.

As described in more detail above, the tube 5 may comprise a first increasing gases passage cross-sectional area portion 35 located at or near the first end 10 of the tube 5 and extending from the main body portion 30 and/or a second increasing gases passage cross-sectional area portion 235 located at or near the second end 20 of the tube 5 and extending from the main body portion 30.

Referring to the first connector 100, as shown in FIG. 14, in a direction towards the first end 10 of tube 5 the gases passage of the medical tube assembly 1 increases in gases passage cross-sectional area from an end of the main body portion 30 nearest the first increasing gases passage cross-sectional area portion through the first increasing gases passage cross-sectional area portion 35.

In a direction towards the first end 10 of tube 5 the gases passage of the medical tube assembly 1 decreases in gases passage cross-sectional area defined by the minimum cross-sectional areas of the gases passage from the first increasing gases passage cross-sectional area portion 35 to the tube connection portion 110 of the first connector 100.

In a direction towards the first end 10 of tube the gases passage of the medical tube assembly 1 increases in gases passage cross-sectional area from the tube connection portion 110 of the first connector 100 to the second portion of the first connector 100.

In some examples, the direction towards the first end 10 of tube 5 is from the main body portion 30 of the tube 5 towards the first end 10 of tube 5.

The cross-sectional area of the gases passage of the main body portion of the tube may in some configurations be defined as described elsewhere in the specification.

The cross-sectional area of the gases passage of the increasing gases passage cross-sectional area may in some configurations be defined as described elsewhere in the specification.

Referring to the first connector 100, as shown in FIG. 14, a cross-sectional area of the gases passage of the first increasing gases passage cross-sectional area portion 35 defined by the local minimum cross-sectional area (and optionally any one of, or all of the local minimum cross-sectional area(s)) of the gases passage of the first increasing gases passage cross-sectional area portion (and optionally a minimum local cross-sectional area of the gases passage of an end of the first increasing gases passage cross-sectional area portion away from the main body portion) is greater than a minimum cross-sectional area of the tube connection portion 110 gases passage of the first connector 100.

A minimum cross-sectional area of the gases passage of the second portion (for example the associated connector portion 70 or the intermediate portion 181) of the first connector 100 is greater than a minimum cross-sectional area of the gases passage of the tube connection portion 110 of the first connector 100.

FIG. 14 shows a minimum diameter 7 (and also a minimum local diameter) of the gases flow path of the tube being larger than a minimum diameter 111 of gases passage of the tube connection portion 110. In the example of FIG. 14, the diameter 41 of the cuff region 40 of the tube is additionally larger than a minimum diameter 111 of gases passage of the tube connection portion 110

FIG. 14 also shows a minimum diameter 111 of the of the gases passage of the tube connection portion 110 being smaller than a minimum diameter of the gases passage of the second portion (being one or both of a minimum diameter 181 of the gases passage of the intermediate portion 183, and a minimum diameter 71 of the gases passage of the associated connector portion 271) of the first connector.

Referring now to the second connector 200, in a direction towards the second end 20 of tube 5 the gases passage of the medical tube assembly 1 increases in gases passage cross-sectional area from an end of the main body portion 30 nearest the second increasing gases passage cross-sectional area portion through the second increasing gases passage cross-sectional area portion 235.

In a direction towards the second end 20 of tube 5 the gases passage of the medical tube assembly 1 decreases in gases passage cross-sectional area from the second increasing gases passage cross-sectional area portion 235 to the tube connection portion 210 of the second connector 200.

In a direction towards the second end 20 of tube 5 the gases passage of the medical tube assembly 1 increases in gases passage cross-sectional area from the tube connection portion 210 of the second connector 200 to the second portion of the second connector 200.

In some examples, the direction towards the second end 20 of tube 5 is from the main body portion 30 of the tube 5 towards the second end 20 of tube 5.

A cross-sectional area of the gases passage of the second increasing gases passage cross-sectional area portion, defined in this example by a local minimum cross-sectional area of the gases passage of the second increasing gases passage cross-sectional area portion, (and optionally a local minimum cross-sectional area of the gases passage of an end of the second increasing gases passage cross-sectional area portion away from the main body portion) is greater than a minimum cross-sectional area of the gases passage of the tube connection portion 210 of the second connector. It will be appreciated that all local minimum cross-sectional area(s) of the gases passage of the second increasing gases passage cross-sectional area portion may be greater than a minimum cross-sectional area of the gases passage of the tube connection portion 210 of the second connector.

The minimum cross-sectional area of the gases passage of the second portion of the second connector 200 is greater than a minimum cross-sectional area of the gases passage of the tube connection portion 210 of the second connector 200. This may be shown for example with respect to FIG. 14 where the tube is circular, which does not show areas, but the diameters are proportional to the area.

FIG. 14 shows a diameter of the gases flow path of the tube 241 (and in this example the cuff region 240 of the tube) being larger than a minimum internal diameter 211 of the tube connection portion 210.

FIG. 14 also shows a minimum diameter 211 of the of gases passage of the tube connection portion 210 being smaller than a minimum diameter of the gases passage of the second portion of the second connector 200 (being a diameter 271 of the associated connector portion 270 of second connector 200).

The associated connector(s) 300 (for example as first and second associated connectors) may be the same type of connector or different types of connector to each other.

In some configurations the first and second associated connectors may be part of one or more tube assemblies, or an apparatus (for example a humidifier, flow generator and/or a ventilator). For example, the first associated connector may be part of a ventilator, and the second associated connector may be connected to a Y-piece and/or patient interface.

The first and second associated connectors may comprise a taper fitting for connection with the first and second connectors 100, 200 respectively.

FIG. 15 shows various examples of cross-sections of the tube 5 being corrugated and the associated cross-sections.

The cross-sectional areas shown in FIGS. 16 and 17 are shown in corrugated portions of the tube and show an example cross-sectional area of the tube. However, it will be appreciated that the cross-sectional area of the corrugated tube and/or the increasing gases passage cross-sectional area portion 35, 25 in some configurations may be defined in a plurality of ways (as described in more detail above).

FIGS. 16 and 17 show the gases passage cross-sectional area as a minimum cross-sectional area defined by an inner surface of the troughs of the tube 5.

FIG. 16 shows a cross-section of the main body portion 30 of the tube 5, defining a cross-sectional area 31 of the main body portion of the tube.

FIG. 17 shows a cross-section of the increasing gases passage cross-sectional area portion 35 of the tube. The cross-section of FIG. 17 is taken as a minimum cross-sectional area 36 of the gases passage at a location where it is increasing towards a first end 10 of the tube 5. The gases passage cross-sectional area may continue to increase for example in the manner shown in FIG. 20.

FIG. 18 shows a cross-section of the tube connection portion 110 of the first connector 100. FIG. 18 shows the cross-sectional area 77 of the tube connection portion 110 of the first connector 100.

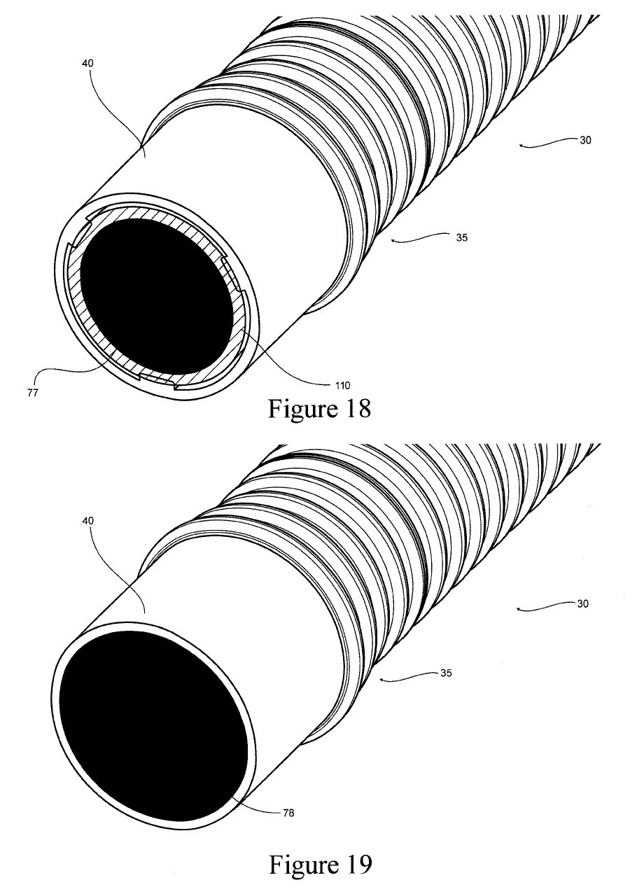

FIG. 19 shows a cross-section of the cuff region 40 of the first connector 100. FIG. 19 shows the cross-sectional area 78 of the cuff region 40 of the first connector 100.

Referring now to FIGS. 23 and 24, in some configurations the cross-sectional area of a gases passage of the medical tube assembly defined by the minimum cross-sectional areas of the gases passage along the length of the tube is equal to or greater than a minimum cross-sectional area of a gases passage of the opening of the associated connector 300. This ensures that the medical tube assembly is not providing any local flow restriction to increase overall resistance to flow of the breathing circuit.

In the examples of FIGS. 23 and 24 the associated connector 300 is configured to engage (and optionally seal with) with an internal surface of the connector 200 (for example associated connector portion 270 of the connector 200). Additionally, or alternatively, in some examples, the associated connector 300 may be configured to engage (and optionally seal with) with an external surface of the connector 200 (for example associated connector portion 270 of the connector 200).

The cross-sectional area of the gases passage of the opening may be the minimum internal cross-sectional area of the opening.

A cross-sectional area of the gases passage of the tube, which in this example is defined by the minimum cross-sectional areas of the gases passage along the length of the main body portion of the tube, is equal to or greater than the cross-sectional area of the gases passage of opening of the associated connector 300. As shown in FIG. 23, a minimum diameter of the gases passage of the tube, which in this example is defined by the minimum cross-sectional areas of the gases passage along the length of the main body portion of the tube is equal to or greater than the diameter of the gases passage of the opening of the associated connector 300.

A minimum cross-sectional area of the gases passage of the connector 200 is equal to or greater than a cross-sectional area of the gases passage of the opening of the associated connector 300.

As shown in FIG. 23, a minimum diameter 211 of the gases passage of the connector 200 is equal to or greater than a diameter 212 of the gases passage of the opening of the associated connector 300. When the cross-sectional area is defined by a diameter the minimum diameter of the minimum cross-sectional area of the gases passage of the connector 100 and the cross-sectional area of the tube 5 may be equal to or greater than a diameter of the gases passage of the opening of the associated connector 300.

In some configurations, the second connector 200 may be configured to connect to a second associated connector 300. In these configurations, one or more of:
a cross-sectional area of the gases passage of the main body portion of the tube,
a minimum cross-sectional area of the gases passage of the first connector, and
a minimum cross-sectional area of the gases passage of the second connector, are equal to or greater than a cross-sectional area of the opening of the second associated connector.

The cross-sectional area of the gases passage of the main body portion 30 of the tube may be defined as described above. In some configurations the main body portion 30 may be the entire tube (for example the tube may not have increasing gases passage cross-sectional area portions).

It will be appreciated that the description above with respect to FIG. 23 may be equally applicable to the connector 100 by itself, the connector 200 by itself, or both the connector 100 and the connector 200.

The minimum diameter 111 of the of the gases passage of the tube connection portion 110 of the first connector 100 may be about 10 mm to about 16 mm, or about 12 mm to about 14 mm, or about 13 mm.

The minimum cross-sectional area of the of the gases passage of the tube connection portion 110 of the first connector 100 may be about 100 mm² to about 150 mm², or about 120 mm² to about 140 mm², or about 130 mm².

The minimum diameter 181 of the gases passage of the intermediate portion 183 of the first connector 100 may be about 10 mm to about 16 mm, or about 12 mm to about 14 mm, or about 13.5 mm.

The minimum cross-sectional area of the gases passage of the intermediate portion 183 of the first connector 100 may be about 120 mm² to about 170 mm², or about 130 mm² to about 150 mm², or about 143 mm².

The minimum diameter 71 of the gases passage of the associated connector portion 71 of the first connector 100 may be about 20 mm to about 25 mm, or about 20 mm to about 23 mm, or about 22.3 mm.

The minimum cross-sectional area of the gases passage of the associated connector portion 71 of the first connector 100 may be about 350 mm² to about 450 mm², or about 380 mm² to about 400 mm², or about 393 mm².

The minimum internal diameter 211 of the tube connection portion 210. of the second connector 200 may be about 10 mm to about 16 mm, or about 11 mm to about 15 mm, or about 12.65 mm.

The minimum internal diameter 211 of the tube connection portion 210. of the second connector 200 may be about 100 mm² to about 150 mm², or about 115 mm² to about 135 mm², or about 125 mm².

The minimum diameter 71 of the gases passage of the associated connector portion 271 of the second connector 200 may be about 10 mm to about 20 mm, or about 14 mm to about 16 mm, or about 15.5 mm.

The minimum diameter 71 of the gases passage of the associated connector portion 271 of the second connector 200 may be about 120 mm² to about 230 mm², or about 180 mm² to about 200 mm², or about 187 mm².

Although embodiments have been described with reference to a number of illustrative embodiments thereof, it will be understood by those skilled in the art that various changes in form and details may be made therein without departing from the spirit and scope of the invention as defined by the appended claims. Therefore, the preferred embodiments should be considered in a descriptive sense only and not for purposes of limitation, and also the technical scope of the invention is not limited to the embodiments. Furthermore, the present invention is defined not by the detailed description of the invention but by the appended claims, and all differences within the scope will be construed as being comprised in the present disclosure.

Where in the foregoing description reference has been made to elements or integers having known equivalents, then such equivalents are included as if they were individually set forth.

Many modifications will be apparent to those skilled in the art without departing from the scope of the present invention as herein described with reference to the accompanying drawings.

1-151. (canceled)

152. A medical tube assembly, the medical tube assembly comprising:
a tube, the tube defining a gases passage extending from a first end of the tube to a second end of the tube, and wherein the tube comprises a water absorbing material, and
a connector defining a gases passage extending through the connector, the connector configured to connect to the first end of the tube,
wherein the tube comprises a main body portion and an increasing gases passage cross-sectional area portion, the increasing gases passage cross-sectional area portion at or near the first end of the tube and extending from the main body portion.

153. The medical tube assembly of claim 152, wherein the connector comprises a tube connection portion provided inside the tube when the connector is connected to the tube.

154. The medical tube assembly of claim 153, wherein when the tube is connected at the tube connection portion to the connector and an end of the increasing gases passage cross-sectional area portion is located at an end of the tube connection portion.

155. The medical tube assembly of claim 154, wherein the medical tube assembly comprises a collar, wherein the collar is configured to engage an external surface of the tube when the connector is connected to the tube, and preferably wherein the tube comprises a cuff region and the cuff region of the tube is retained between the collar and the tube connection portion.

156. The medical tube assembly of claim 155, wherein the connector and/or the collar comprise at least one attachment feature, the at least one attachment feature configured to retain the collar to the connector, the tube connection portion of the connector comprising a retention feature, the retention feature configured to engage with an internal surface of the tube to aid in retaining the tube on the connector, and the tube connection portion and the collar comprising a sealing feature, the sealing feature configured to provide a pneumatic seal between the collar and the tube connection portion, wherein the sealing feature is located between the retention feature and the at least one attachment feature.

157. The medical tube assembly of claim 155, wherein the main body portion of the tube is corrugated.

158. The medical tube assembly of claim 155, wherein the main body portion comprises at least one corrugation, the at least one corrugation of the main body portion of the tube comprising alternating crests and troughs.

159. The medical tube assembly of any one of claim 158, wherein the increasing gases passage cross-sectional area portion of the main body portion of the tube is defined by:
a) a minimum cross-sectional area of the gases passage of the main body portion of the tube, or
b) a maximum cross-sectional area of the gases passage of the main body portion of the tube, or
c) when the tube is corrugated, a nominal cross-sectional area being an average of the minimum cross-sectional area of the gases passage of the main body portion of the tube and the maximum cross-sectional area of the gases passage of the main body portion of the tube.

160. The medical tube assembly of any one of claim 156, wherein the medical tube assembly comprises at least one heater wire.

161. The medical tube assembly of claim 160, wherein the at least one heater wire is located in a wall of the tube, around the tube, or within the gases passage of the tube and/or the gases passage of the connector, and preferably wherein the medical tube assembly comprises an electrical connector configured to allow for electrical connection to the at least one heater wire.

162. The medical tube assembly of any one of claim 152, wherein the increasing gases passage cross-sectional area portion of the tube comprises an increasing cross-sectional area of the gases passage of the tube in a direction from the main body portion of the tube towards the first end of the tube.

163. The medical tube assembly of any one of claim 162, wherein the increasing gases passage cross-sectional area portion of the tube is corrugated.

164. The medical tube assembly of any one of claim 162, wherein the increasing gases passage cross-sectional area portion is defined by a number of corrugations and preferably wherein the number of corrugations is about 1 to about 10 corrugations, or about 2 to about 7 corrugations, or about 2 to about 5 corrugations, or about 2 to about 2.5 corrugations, or about 2 corrugations.

165. The medical tube assembly of any one of claim 162, wherein the increasing gases passage cross-sectional area portion is a flared portion.

166. The medical tube assembly of any one of claim 162, wherein the water absorbing material of the tube is a breathable material.
